(19) United States
(12) Patent Application Publication
(10) Pub. No.: US 2004/0106540 A1
(43) Pub. Date: Jun. 3, 2004

(54) Method of treating cancer

(76) Inventors: Stanley F Barnett, North Wales, PA (US); Deborah DeFeo-Jones, Lansdale, PA (US); Kathleen M Haskell, Colmar, PA (US); Hans E Huber, Lansdale, PA (US); Deborah D Nahas, Perkasie, PA (US)

Correspondence Address: MERCK AND CO INC, P O BOX 2000, RAHWAY, NJ, 070650907

(21) Appl. No.: 10/473,791

(22) PCT Filed: Apr. 8, 2002
(86) PCT No.: PCT/US02/10879

(51) Int. Cl. A61K 38/00

(52) U.S. Cl. 514/2

(57) ABSTRACT

The present invention is directed to a method of treating cancer which comprises administration of a compound which selectively inhibits the activity of one or two of the isoforms of Akt, a serine/threonine protein kinase. The invention is particularly directed to the method wherein the compound is dependent on the presence of the plestrin homology domain of Akt for its inhibitory activity.

### BACKGROUND OF THE INVENTION

The present invention relates to methods of treating cancer by selectively inhibiting one or more isoforms of Akt (also known as PKB, and referred to herein as either Akt or Akt/PKB). The present invention also relates to a method of identifying such compounds.

Apoptosis (programmed cell death) plays essential roles in embryonic development and pathogenesis of various diseases, such as degenerative neuronal diseases, cardiovascular diseases and cancer. Recent work has led to the identification of various pro- and anti-apoptotic gene products that are involved in the regulation or execution of programmed cell death. Expression of anti-apoptotic genes, such as Bcl2 or Bcl-x_{L}, inhibits apoptotic cell death induced by various stimuli. On the other hand, expression of pro-apoptotic genes, such as Bax or Bad, leads to programmed cell death (Aams et al. Science, 281:1322-1326 (1998)). The execution of programmed cell death is mediated by caspase-1 related proteinases, including caspase-3, caspase-7, caspase-8 and caspase-9 etc (Thomeberry et al. Science, 281:1312-1316 (1998)).

The phosphatidylinositol 3′-OH kinase (PI3K)/Akt/PKB pathway appears important for regulating cell survival/cell death (Kulik et al. Mol. Cell. Biol. 17:1595-1606 (1997); Franke et al, Cell, 88:435-437 (1997); Kauffmann-Zeh et al. Nature 385:544-548 (1997) Hemmings Science, 275:628-630 (1997); Dudek et al., Science, 275:661-665 (1997)). Survival factors, such as platelet derived growth factor (PDGF), nerve growth factor (NGF) and insulin-like growth factor-1 (IGF-1), promote cell survival under various conditions by inducing the activity of PI3K (Kulik et al. 1997, Hemmings 1997). Activated PI3K leads to the production of phosphatidylinositol (3,4,5)-triphosphate (Ptdlns(3,4,5)-P3), which in turn binds to, and promotes the activation of, the serine/threonine kinase Akt, which contains a pleckstrin homology (PH)-domain (Franke et al Cell, 81:727-736 (1995); Hemmings Science, 277:534 (1997); Downward, Curr. Opin. Cell Biol. 10:262-267 (1998), Alessi et al., EMBO J. 15: 6541-6551 (1996)). Specific inhibitors of PI3K or dominant negative Akt/PKB mutants abolish survival-promoting activity of these growth factors or cytokines. It has been previously disclosed that inhibitors of PI3K (LY294002 or wortmannin) blocked the activation of Akt/PKB by upstream kinases. In addition, introduction of constitutively active PI3K or Akt/PKB mutants promotes cell survival under conditions in which cells normally undergo apoptotic cell death (Kulik et al. 1997, Dudek et al. 1997). Analysis of Akt levels in human tumors showed that Akt-2 is overexpressed in a significant number of ovarian (J. Q. Cheung et al. Proc. Natl. Acad. Sci. U.S.A. 89:9267-9271(1992)) and pancreatic cancers (J. Q. Cheung et al. Proc. Natl. Acad. Sci. U.S.A. 93:3636-3641 (1996)). Similarly, Akt3 was found to be overexpressed in breast and prostate cancer cell lines (Nakatani et al. J. Biol. Chem. 274:21528-21532 (1999).

The tumor suppressor PTEN; a protein and lipid phosphatase that specifically removes the 3′ phosphate of PtdIns(3,4,5)-P3, is a negative regulator of the PI3K/Akt pathway (Li et al. Science 275:1943-1947 (1997), Stambolic et al. Cell 95:29-39 (1998), Sun et al. Proc. Natl. Acad. Sci. U.S.A. 96:6199-6204 (1999)). Germline mutations of PTEN are responsible for human cancer syndromes such as Cowden disease (Liaw et al. Nature Genetics 16:64-67 (1997)). PTEN is deleted in a large percentage of human tumors and tumor cell lines without functional PTEN show elevated levels of activated Akt (Li et al. supra, Guldberg et al. Cancer Research 57:3660-3663 (1997), Risinger et al. Cancer Research 57:4736-4738 (1997)).

These observations demonstrate that the PI3K/Akt pathway plays important roles for regulating cell survival or apoptosis in tumorigenesis.

Three members of the Akt/PKB subfamily of second-messenger regulated serine/threonine protein kinases have been identified and termed Akt1/PKBα, Akt2/PKBβ, and Akt3/PKBγ respectively. The isoforms are homologous, particularly in regions encoding the catalytic domains. Akt/PKBs are activated by phosphorylation events occurring in response to PI3K signaling. PI3K phosphorylates membrane inositol phospholipids, generating the second messengers phosphatidylinositol 3,4,5-trisphosphate and phosphatidylinositol 3,4-bisphosphate, which have been shown to bind to the PH domain of Akt/PKB. The current model of Akt/PKB activation proposes recruitment of the enzyme to the membrane by 3′-phosphorylated phosphoinositides, where phosphorylation of the regulatory sites of Akt/PKB by the upstream kinases occurs (B. A. Hemmings, Science 275:628-630 (1997); B. A. Hemmings, Science 276:534 (1997); J. Downward, Science 279:673-674 (1998)).

Phosphorylation of Akt1/PKBα occurs on two regulatory sites, Thr³⁰⁸ in the catalytic domain activation loop and on Ser⁴⁷³ near the carboxy terminus (D. R. Alessi et al. EMBO J. 15:6541-6551 (1996) and R. Meier et al. J. Biol. Chem. 272:30491-30497 (1997)). Equivalent regulatory phosphorylation sites occur in Akt2/PKβ and Akt3/PYBγ. The upstream kinase, which phosphorylates Akt/PKB at the activation loop site has been cloned and termed 3′-phosphoinositide dependent protein kinase 1 (PDK1). PDK1 phosphorylates not only Akt/PKB, but also p70 ribosomal S6 kinase, p90RSK, serum and glucocorticoid-regulated kinase (SGK), and protein kinase C. The upstream kinase phosphorylating the regulatory site of Akt/PKB near the carboxy terminus has not been identified yet, but a recent report implies a role for the integrin-linked kinase (ILK-1), a serine/threonine protein kinase, or autophosphorylation.

Inhibition of Akt activation and activity can be achieved by inhibiting PI3K with inhibitors such as LY294002 and wortmannin. However, PI3K inhibition has the potential to indiscriminately affect not just all three Akt isozymes but also other PH domain-containing signaling molecules that are dependent on PdtIns(3,4,5)-P3, such as the Tec family of tyrosine kinases. Furthermore, it has been disclosed that Akt can be activated by growth signals that are independent of PI3K.

Alternatively, Akt activity can be inhibited by blocking the activity of the upstream kinase PDK1. No specific PDK1 inhibitors have been disclosed. Again, inhibition of PDK1 would result in inhibition of multiple protein kinases whose activities depend on PDK1, such as atypical PKC isoforms, SGK, and S6 kinases (Williams et al. Curr. Biol. 10:439-448 (2000).

It is therefore an object of the instant invention to provide a method for treating cancer that comprises administering an inhibitor of Akt/PKB activity that selectively inhibits one or more of the Akt/PKB isoforms over the other isoform(s).

It is also an object of the present invention to provide a method for treating cancer that comprises administering an inhibitor of Akt/PKB activity that selectively inhibits one or more of the Akt/PKB isoforms and is dependent on the PH domain, the hinge region of the protein or both the PH domain and the hinge region for its inhibitory activity.

It is also an object of the instant invention to provide a method of identifying an inhibitor of PKB that selectively inhibits one or more of the Akt/PKB isoforms and is dependent on the PH domain for its inhibitory activity.

### SUMMARY OF THE INVENTION

The instant invention provides for a method of treating cancer which comprises administering to a mammal an inhibitor of Akt/PKB activity that selectively inhibits one or more of the Akt/PKB isoforms. The invention also provides for a method of inhibiting Akt/PKB activity by administering a compound that is an inhibitor of Akt/PKB activity that selectively inhibits one or more of the Akt/PKB isoforms and is dependent on the PH domain for its inhibitory activity. A method of identifying such selective inhibitors of Akt/PKB activity is also disclosed.

### DETAILED DESCRIPTION OF THE INVENTION

The present invention relates to a method of inhibiting Akt/PKB activity which comprises administering to a mammal in need thereof a pharmaceutically effective amount of a compound that selectively inhibits one or more of the Akt/PKB isoforms. The invention also relates to a method of treating cancer that comprises administering to a mammal in need thereof an inhibitor whose activity is dependent on the presence of the pleckstrin homology (PH) domain, the hinge region or both the PH domain and the hinge region of Akt.

Direct inhibition of one or more Akt isozymes provides the most specific means of regulating the PI3K/Akt pathway.

The term “inhibiting Akt/PKB activity” as used herein describes the decrease in the in vitro and in vivo biochemical modifications resulting from the phosphorylation of Akt by upstream kinases and/or the subsequent phosphorylation of downstream targets of Akt by activated Akt. Thus, the terms “inhibitor of Akt/PKB activity” and “inhibitor of Akt/PKB [isoforms]” describe an agent that, by binding to Akt, either inhibits the phosphorylation of Akt by upstream kinases (thereby reducing the amount of activated Akt) or inhibits the phosphorylation by activated Akt of protein targets of Akt, or inhibits both of these biochemical steps. In a preferred embodiment, the inhibitor utilized in the instant methods inhibits the phosphorylation of Akt by upstream kinases (thereby reducing the amount of activated Akt) and inhibits the phosphorylation by activated Akt of protein targets of Akt.

Preferably, the selective inhibitor useful in the instant method of treatment is selected from: a selective inhibitor of Akt 1, a selective inhibitor of Akt 2 or a selective inhibitor of both Akt 1 and Akt 2.

In a sub-embodiment, the selective inhibitor useful in the instant method of treatment is selected from: a selective inhibitor of Akt 1, a selective inhibitor of Akt 2, a selective inhibitor of Akt3 or a selective inhibitor of two of the three Akt isoforms.

The term “selective inhibitor” as used herein is intended to mean that the inhibiting compound exhibits greater inhibition against the activity of the indicated isoform(s) of Akt, when compared to the compounds inhibition of the activity of the other Akt isoform(s) and other kinases, such as PKA and PKC. Preferably, the selectively inhibiting compound exhibits at least about a 5 fold greater inhibition against the activity of the indicated isoform(s) of Akt. More preferably, the selectively inhibiting compound exhibits at least about a 50 fold greater inhibition against the activity of the indicated isoform(s) of Akt.

In a second embodiment of the invention, the methods of treating cancer and inhibiting Akt comprise administering an inhibitor whose activity is dependent on the presence of the pleckstrin homology (PH) domain, the hinge region or both the PH domain and the hinge region of Akt.

The PH domains and hinge regions of the three Akt isoforms, though presumably functionally equivalent in terms of lipid binding, show little sequence homology and are much less conserved than the catalytic domains. Inhibitors of Akt that function by binding to the PH domain, the hinge region or both are thus able to discriminate between the three Akt isozymes.

A selective inhibitor whose inhibitory activity is dependent on the PH domain exhibits a decrease in in vitro inhibitory activity or no in vitro inhibitory activity against truncated Akt/PKB proteins lacking the PH domain.

A selective inhibitor whose inhibitory activity is dependent on the hinge region, the region of the proteins between the PH domain and the kinase domain (see Konishi et al. Biochem. and Bioplzys. Res. Comm. 216: 526-534 (1995), FIG. 2, incorporated herein by reference), exhibits a decrease in in vitro inhibitory activity or no in vitro inhibitory activity against truncated Aict proteins lacking the PH domain and the hinge region or the hinge region alone.

The method of using such an inhibitor that is dependent on either the PH domain, the hinge region or both provides a particular advantage since the PH domains and hinge regions in the Akt isoforms lack the sequence homology that is present in the rest of the protein, particularly the homology found in the kinase domains (which comprise the catalytic domains and ATP-binding consensus sequences). It is therefore observed that certain inhibitor compounds, such as those described herein, are not only selective for one or two isoforms of Akt, but also are weak inhibitors or fail to inhibit other kinases, such as PKA and PKC, whose kinase domains share some sequence homology with the kinase domains of the Akt/PKB isoforms. Both PKA and PKC lack a PH domain and a hinge region.

Preferably, the selective inhibitor of the second embodiment is selected from: a selective inhibitor of Akt 1, a selective inhibitor of Akt 2 or a selective inhibitor of both Akt 1 and Akt 2.

In a sub-embodiment of the second embodiment, the selective inhibitor useful in the instant method of treatment is selected from: a selective inhibitor of Akt 1, a selective inhibitor of Akt 2, a selective inhibitor of Akt3 or a selective inhibitor of two of the three Akt isoforms.

In another sub-embodiment, the selective inhibitor of one or two of the Akt isoforms useful in the instant method of treatment is not an inhibitor of one or both of such Akt isoforms that have been modified to delete the PH domain, the hinge region or both the PH domain and the hinge region.

In another sub-embodiment, the selective inhibitor of all three Akt isoforms useful in the instant method of treatment is not an inhibitor of one, two or all of such Akt isoforms that have been modified to delete the PH domain, the hinge region or both the PH domain and the hinge region.

The present invention further relates to a method of identifying a compound that is a selective inhibitor of one or two of the Akt/PKB isoforms, or all three isoforms, whose inhibitory efficacy is dependent on the PH domain. The method comprises the steps of:

a) determining the efficacy of a test compound in inhibiting the activity of an Akt isoforn;

b) determining the efficacy of the test compound in inhibiting the activity of the Akt isoform that has been modified to delete the PH domain; and

c) comparing the activity of the test compound against the Akt isoform with the activity of the test compound against the modified Akt isoform lacking the PH domain.

The present invention also relates to a method of identifying a compound that is a selective inhibitor of one or two of the Akt/PKB isoforms, or all three isoforms, whose inhibitory efficacy is dependent on the hinge region. The method comprises the steps of:

a) determining the efficacy of a test compound in inhibiting the activity of an Akt isoform;

b) determining the efficacy of the test compound in inhibiting the activity of the Akt isoform that has been modified to delete the PH domain;

c) determining the efficacy of the test compound in inhibiting the activity of the Akt isoform that has been modified to delete the PH domain and the hinge region; and

d) comparing the activity of the test compound against the Akt isoform, the activity of the test compound against the modified Akt isoform lacking the PH domain, and the activity of the test compound against the modified Akt isoform lacking the PH domain and the hinge region.

The compounds that are identified by the methods described above as inhibitors of the activity of one or more Akt isoforms that are dependent on the presence of either or both the PH domain or hinge region of the Akt isoform will be useful in the methods of treatment disclosed herein. Such compounds may further be useful as components in assay systems that may be used to identify other inhibitors of the activity of one or more Akt isoforms wherein the other inhibitors have inhibitory activity through selective binding and/or interaction with the kinase region of the Akt isoform(s). Particularly useful as an assay component would be a PH domain and/or hinge region dependent inhibitor that is an irreversible inhibitor of the Akt isoform(s). Methods are well known in the art for determining whether the activity of an inhibitor of a biological activity or enzyme is reversible or irreversible.

It is understood that the modified Akt isoforms useful in the above methods of identification may alternatively lack less than the full PH region and/or hinge region. For example, a modified Akt isoform may lack the full PH domain and a portion of the hinge region. It is also understood that the methods may alternatively comprise modified Akt isoforms wherein the PH domain and/or the hinge region are modified by a specific point mutation(s) in those amino acid sequences. Such a method comprising a modified Akt isoform having a point mutatibn(s) in the PH domain and/or the hinge region may not only identify whether the activity of an inhibitor compound is dependent on the presence of the PH domain and/or the hinge region, but may also identify the specific site in the Akt isoform where the inhibitor compound interacts or binds with the protein.

The present invention is also directed to the cloning and expression of modified versions of the Akt isoforms that are useful in the methods of identifying inhibitor compounds described hereinabove. Specifically, modified Akt isoforms lacking only the PH domain (deletion of about aa 4-110 for Akt 1, deletion of about aa 4-110 for Akt 2 and deletion of about aa 4-109 for Akt 3) may be prepared by techniques well known in the art. Similarly, modified Akt isoforms wherein both the PH domain and the hinge region are deleted (deletion of about aa 4-145 for Akt 1, deletion of about aa 4-147 for Akt 2 and deletion of about aa 4-143 for Akt 3) may be prepared by techniques well known in the art.

The present invention is further directed to the cloning and expression of modified versions of the Akt isoforms wherein one or more point mutations are made to the amino acid sequences of the PH domain and the hinge region. Preferably, one or two point mutations are made to the amino acid sequences of the PH domain and the hinge region. Most preferably, one point mutation is made to the amino acid sequences of the PH domain and the hinge region.

The methods of the instant invention are useful in the treatment of cancer, in particular cancers associated with irregularities in the activity of Akt and/or GSK3. Such cancers include, but are not limited to ovarian, pancreatic and breast cancer.

The compounds of this invention may be administered to mammals, preferably humans, either alone or, preferably, in combination with pharmaceutically acceptable carriers, excipients or diluents, in a pharmaceutical composition, according to standard pharmaceutical practice. The compounds can be administered orally or parenterally, including the intravenous, intramuscular, intraperitoneal, subcutaneous, rectal and topical routes of administration.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, cellulose acetate buryrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavouring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of Formula A may also be administered in the form of a suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of Formula A are employed. (For purposes of this application, topical application shall include mouth washes and gargles.)

The compounds useful in the instant method of treatment of the present invention can be administered in ifitranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

As used herein, the term “composition” is intended to encompass a product comprising the specified ingredients in the specific amounts, as well as any product which results, directly or indirectly, from combination of the specific ingredients in the specified amounts.

The compounds identified by the instant method may also be co-administered with other well known therapeutic agents that are selected for their particular usefulness against the condition that is being treated. For example, the instant compounds may be useful in combination with known anti-cancer and cytotoxic agents. Similarly, the instant compounds may be useful in combination with agents that are effective in the treatment and prevention of neurofibromatosis, restinosis, polycystic kidney disease, infections of hepatitis delta and related viruses and fungal infections. The instant compositions may also be useful in combination with other inhibitors of parts of the signaling pathway that links cell surface growth factor receptors to nuclear signals initiating cellular proliferation. Thus, the instant compounds may be utilized in combination with inhibitors of prenyl-protein transferase, including protein substrate competitive inhibitors of farnesyl-protein transferase, farnesyl pyrophosphate competitive inhibitors of the activity of farnesyl-protein transferase and/or inhibitors of geranylgeranyl-protein transferase. The instant compositions may also be co-administered with compounds that are selective inhibitors of geranylgeranyl protein transferase or selective inhibitors of farnesyl-protein transferase. The instant compositions may also be administered in combination with a compound that has Raf, MEK or Map kinase antagonist activity.

The compounds useful in the method of treatment of the instant invention may also be co-administered with other well known cancer therapeutic agents that are selected for their particular usefulness against the condition that is being treated. Included in such combinations of therapeutic agents are combinations with an antineoplastic agent. It is also understood that the instant compositions and combinations may be used in conjunction with other methods of treating cancer and/or tumors, including radiation therapy and surgery.

Additionally, compositions useful in the method of treatment of the instant invention may also be useful as radiation sensitizers. Radiation therapy, including x-rays or gamma rays that are delivered from either an externally applied beam or by implantation of tiny radioactive sources, may also be used in combination with the compounds of the instant invention.

If formulated as a fixed dose, such combination products employ the combinations of this invention within the dosage range described below and the other pharmaceutically active agent(s) within its approved dosage range. Combinations of the instant invention may alternatively be used sequentially with known pharmaceutically acceptable agent(s) when a multiple combination formulation is inappropriate.

Radiation therapy, including x-rays or gamma rays that are delivered from either an externally applied beam or by implantation of tiny radioactive sources, may also be used in combination with an inhibitor of prenyl-protein transferase alone to treat cancer.

The instant compositions may also be useful in combination with an integrin antagonist for the treatment of cancer, as described in U.S. Ser. No. 09/055,487, filed Apr. 6, 1998, which is incorporated herein by reference.

As used herein the term an integrin antagonist refers to compounds which selectively antagonize, inhibit or counteract binding of a physiological ligand to an integrin(s) that is involved in the regulation of angiogenisis, or in the growth and invasiveness of tumor cells. In particular, the term refers to compounds which selectively antagonize, inhibit or counteract binding of a physiological ligand to the αvβ3 integrin, which selectively antagonize, inhibit or counteract binding of a physiological ligand to the αvβ5 integrin, which antagonize, inhibit or counteract binding of a physiological ligand to both the αvβ3 integrin and the αvβ5 integrin, or which antagonize, inhibit or counteract the activity of the particular integrin(s) expressed on capillary endothelial cells. The term also refers to antagonists of the αvβ6, αvβ8, α1β1, α2β1, α5β1, α6β1 and α6β4 integrins. The term also refers to antagonists of any combination of αvβ3, αvβ5, αvβ6, αvβ8, α1β1, α2β1, α5β1, α6β1 and α6β4 integrins. The instant compounds may also be useful with other agents that inhibit angiogenisis and thereby inhibit the growth and invasiveness of tumor cells, including, but not limited to angiostatin and endostatin.

When a composition according to this invention is administered into a human subject, the daily dosage will normally be determined by the prescribing physician with the dosage generally varying according to the age, weight, and response of the individual patient, as well as the severity of the patient's symptoms.

In one exemplary application, a suitable amount of an inhibitor of one or two of the Akt/PKB isoforms is administered to a mammal undergoing treatment for cancer. Administration occurs in an amount of inhibitor of between about 0.1 mg/kg of body weight to about 60 mg/kg of body weight per day, preferably of between 0.5 mg/kg of body weight to about 40 mg/kg of body weight per day. A particular therapeutic dosage that comprises the instant composition includes from about 0.01 mg to about 1000 mg of inhibitor of one or two of the Akt/PKB isoforms. Preferably, the dosage comprises from about 1 mg to about 1000 mg of inhibitor of one or two of the Akt/PKB isoforms.

Examples of an antineoplastic agent include, in general, microtubule-stabilising agents ( such as paclitaxel (also known as Taxol®), docetaxel (also known as Taxotere®), or their derivatives); alkylating agents, anti-metabolites; epidophyllotoxin; an antineoplastic enzyme; a topoisomerase inhibitor; procarbazine; mitoxantrone; platinum coordination complexes; biological response modifiers and growth inhibitors; hormonal/anti-hormonal therapeutic agents and haematopoietic growth factors.

Example classes of antineoplastic agents include, for example, the anthracycline family of drugs, the vinca drugs, the mitomycins, the bleomycins, the cytotoxic nucleosides, the taxanes, the epothilones, discodermolide, the pteridine family of drugs, diynenes and the podophyllotoxins. Particularly useful members of those classes include, for example, doxorubicin, carminomycin, daunorubicin, aminopterin, methotrexate, methopterin, dichloro-methotrexate, mitomycin C, porfiromycin, 5-fluorouracil, 6-mercaptopurine, gemcitabine, cytosine arabinoside, podophyllotoxin or podo-phyllotoxin derivatives such as etoposide, etoposide phosphate or teniposide, melphalan, vinblastine, vincristine, leurosidine, vindesine, leurosine, paclitaxel and the like. Other useful antineoplastic agents include estramustine, cisplatin, carboplatin, cyclophosphamide, bleomycin, gemcitibine, ifosamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, pyridobenzoindole derivatives, interferons and interleukins.

Compounds which are useful in the methods of treatment of the instant invention and are identified by the properties described hereinabove include:

i) a compound of the formula I:

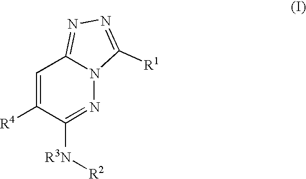

wherein

R¹ represents phenyl, furyl, thienyl or pyridinyl, any of which groups may be optionally substituted with one, two or three substituents, independently selected from:

a) halogen;

b) C₁₋₄ alkyl;

c) C₁₋₄ alkoxy;

d) cyano;

e) di(C₁₋₄ alkyl)amino;

f) hydroxy;

R² represents amino-C₁₋₆ alkyl, C₁₋₄ alkylamino-(C₁₋₆)alkyl, di(C₁₋₄ alkyl)amino-(C₁₋₆)alkyl, hydroxy-(C₁₋₆)alkyl or C₁₋₄ alkoxy-(C₁₋₆)alkyl, any of which groups may be optionally substituted;

R³ represents hydrogen or C₁₋₆ alkyl; and

R⁴ is selected from: C₃₋₇ cycloalkyl and aryl, any of which groups may be optionally substituted;

ii) a compound of the formula II:

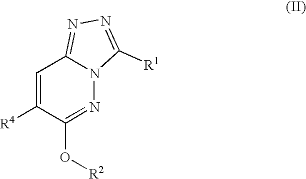

wherein R¹ represents phenyl, furyl, thienyl or pyridinyl, any of which groups may be optionally substituted with one, two or three substituents, independently selected from:

a) halogen;

g) C₁₋₄ alkyl;

h) C₁₋₄ alkoxy;

i) cyano;

j) di(Chd 1-4 alkyl)amino;

k) hydroxy;

R² represents amino-C₁₋₆ alkyl, C₁₋₄ alkylamino-(C₁₋₆)alkyl, di(C₁₋₄ alkyl)amino-(C₁₋₆)alkyl, hydroxy-(C₁₋₄)alkyl or C₁₋₄ alkoxy-(C₁₋₆)alkyl, any of which groups may be optionally substituted; and

R⁴ is selected from: C₃₋₇ cycloalkyl and aryl, any of which groups may be optionally substituted;

iii) a compound of the formula III:

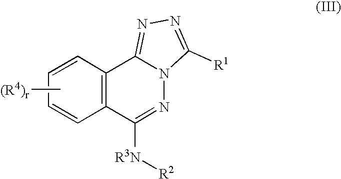

wherein

R¹ represents phenyl, furyl, thienyl or pyridinyl, any of which groups may be optionally substituted with one, two or three substituents, independently selected from:

a) halogen;

l) C₁₋₄ alkyl;

m) C₁₋₄ alkoxy;

n) cyano;

o) di(C₁₋₄ alkyl)amino;

p) hydroxy;

R² represents amino-C₁₋₆ alkyl, C₁₋₆ alkylamnino-(C₁₋₆)alkyl, di(C₁₋₄ alkyl)amino-(C₁₋₆)alkyl, hydroxy-(C₁₋₆)alkyl or C₁₋₄ alkoxy-(C₁₋₆)alkyl, any of which groups may be optionally substituted;

R³ represents hydrogen or C₁₋₆ alkyl; and

R⁴ independently represents hydrogen, C₁₋₄-alkyl, halogen, HO— or C₁₋₆ alkyl-O;

r is 1 or 2;

iv) a compound of the formula IV:

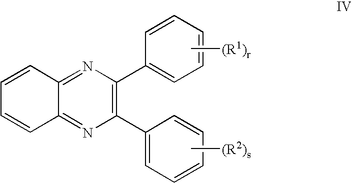

wherein

R¹ independently represents amino, C₁₋₆-alkyl amino, di-C₁₋₆-alkylamino, amino-C₁₋₆₀ alkyl, C₁₋₆ alkylamino-(C₁₋₆)alkyl or di(C,.6 alkyl)amino-(C₁₋₆)alkyl;

R² independently represents hydrogen, amino, C₁₋₆-alkyl amino, di-C₁₋₆-alkylamino, amino-C₁₋₆ alkyl, C₁₋₆ alkylamino-(C₁₋₆)alkyl or di(C₁₋₆ alkyl)amino-(C₁₋₆)alkyl;

r is 1 to 3;

s is 1 to 3;

v) a compound of the formula V:

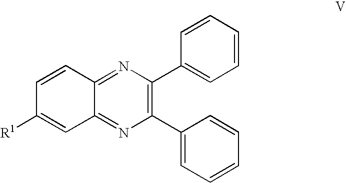

wherein

R¹ independently represents hydrogen, C₁₋₆-alkyl, halogen, HO— or C₁₋₆ alkyl-O; or a pharmaceutically acceptable salt thereof.

As used herein, the expression “C₁₋₄ alkyl” includes methyl and ethyl groups, and straight-chained or branched propyl, butyl, pentyl and hexyl groups. Particular alkyl groups are methyl, ethyl, n-propyl, isopropyl, tert-butyl and 2,2-dimethylpropyl. Derived expressions such as “C₁₋₆ alkoxy” are to be construed accordingly.

As used herein, the expression “C₁₋₄ alkyl” includes methyl and ethyl groups, and straight-chained or branched propyl and butyl groups. Particular alkyl groups are methyl, ethyl, n-propyl, isopropyl and tert-butyl. Derived expressions such as “C₁₋₄ alkoxy” are to be construed accordingly.

Typical C₃₋₇ cycloalkyl groups include cyclopropyl, cyclobutyl, cyclopentyl and cyclohexyl.

The expression “C₃₋₇ cycloalkyl(C₁₋₆)alkyl” as used herein includes cyclopropylmethyl, cyclobutylmethyl, cyclopentylmethyl and cyclohexylmethyl.

Typical C₄₋₇ cycloalkenyl groups include cyclobutenyl, cyclopentenyl and cyclohexenyl.

Typical aryl groups include phenyl and naphthyl, preferably phenyl.

The expression “aryl(C₁₋₆)alkyl” as used herein includes benzyl, phenylethyl, phenylpropyl and naphthylmethyl.

The term “halogen” as used herein includes fluorine, chlorine, bromine and iodine, especially fluorine or chlorine.

For use in medicine, the salts of the compounds of formula I will be. pharmaceutically acceptable salts. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. Furthermore, where the compounds of the invention carry an acidic moiety, suitable pharmaceutically acceptable salts thereof may include alkali metal salts, e.g. sodium or potassium salts; alkaline earth metal salts, e.g. calcium or magnesium salts; and salts formed with suitable organic ligands, e.g. quaternary ammonium salts.

The present invention includes within its scope prodrugs of the compounds of formulae I-V above. In general, such prodrugs will be functional derivatives of the compounds of formulae I-V which are readily convertible in vivo into the required compound of formulae I-V. Conventional procedures for the selection and preparation of suitable prodrug derivatives are described, for example, in Design of Prodrugs, ed. H. Bundgaard, Elsevier, 1985.

Where the compounds useful in the instant methods of treatment have at least one asymmetric center, they may accordingly exist as enantiomers. Where such compounds possess two or more asymmetric centers, they may additionally exist as diastereoisomers. It is to be understood that all such isomers and mixtures thereof in any proportion are encompassed within the scope of the present invention.

Examples of suitable values for the substituent R⁴ include methyl, ethyl, isopropyl, tert-butyl, 1,1-dimethylpropyl, methyl-cyclopropyl, cyclobutyl, methyl-cyclobutyl, cyclopentyl, methyl-cyclopentyl, cyclohexyl, cyclobutenyl, phenyl, pyrrolidinyl, methyl-pyrrolidinyl, piperidinyl, morpholinyl, thiomorpholinyl, pyridinyl, furyl, thienyl, chloro-thienyl and diethylamino.

In a particular embodiment, the substituent R⁴ represents C₃₋₇ cycloalkyl or phenyl, either unsubstituted or substituted by C₁₋₆ alkyl, especially methyl. Favourably, Z represents cyclobutyl or phenyl.

Examples of typical optional substituents on the group R¹ include methyl, fluoro and methoxy.

Representative values of R¹ include cyclopropyl, phenyl, methylphenyl, fluorophenyl, difluorophenyl, methoxyphenyl, furyl, thienyl, methyl-thienyl and pyridinyl.

In a particular embodiment, R² represents amino-C₁₋₆ alkyl, C₁₋₄ alkylamino-(C₁₋₆)alkyl or di(C₁₋₄ alkyl)amino-(C₁₋₆)alkyl. Representative values of R² include but are not limited to dimethylaminomethyl, aminoethyl, dimethylaminoethyl, diethylaminoethyl, 3-dimethylaminopropyl, 3-methylaminopropyl, 3-dimethylamino-2,2-dimethylpropyl and, 3-dimethylamino-2-methylpropyl.

Suitably, R³ represents hydrogen or methyl.

In a particular embodiment of the method of the instant invention, the compound that selectively inhibits one or two of the Akt/PKB isoforms is selected from:

i) a compound of the formula IA:

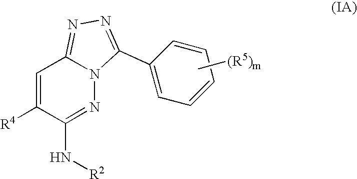

wherein

R² is as defined with reference to formula I above;

R⁴ is selected from: C₃₋₇ cycloalkyl and phenyl, any of which groups may be optionally substituted.

m is 0, 1, 2 or 3; and

R⁵ independently represents halogen, C₁₋₄ alkyl or C₁₋₆ alkoxy;

ii) a compound of the formula IIA:

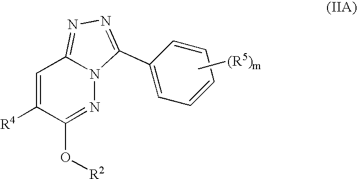

wherein

R² is as defined with reference to formula II above;

R⁴ is selected from: C₃₋₇ cycloalkyl and phenyl, any of which groups may be optionally substituted.

m is 0, 1, 2 or 3; and

R⁵ independently represents halogen, C₁₋₄ alkyl or C₁₋₆ alkoxy;

iii) a compound of the formula IVa:

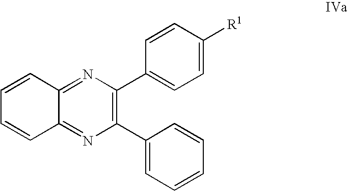

wherein

R¹ independently represents amino, C₁₋₆-alkyl amino, di-C₁₋₆-alkyamino, amino-C₁₋₆ alkyl, C₁₋₆ alkylamino-(C₁₋₆)alkyl or di(C₁₋₆ alkyl)amino-(C₁₋₆)alkyl; or the pharmaceutically acceptable salts thereof.

Specific compounds which are inhibitors of one or two of the Akt/PKB isoforms and are therefore useful in the present invention include:

N′-(7-Cyclobutyl-3-phenyl-[1,2,4]triazolo[4,3-b]pyridazin-6-yl)-2,2, N,N-tetramethyl-propane-1,3-diamine

N′-(7-Cyclobutyl-3-(3,5-difluoro-phenyl)-[1,2,4]triazolo[4,3-b]pyridazin-6-yl)-2,2,N,N-tetramethyl-propane-1,3-diamine

N′-(7-Cyclobutyl-3-(3,4-difluoro-phenyl)-[1,2,4]triazolo[4,3-b]pyridazin-6-yl)-2,2,N,N-tetramethyl-propaner1,3-diamine

N′-(7-Cyclobutyl-3-(4-fluoro-phenyl)-[1,2,4]triazolo[4,3-b]pyridazin-6-yl)-2,2,N,N-tetramethyl-propane-1,3-diamine

N′-(7-Cyclobutyl-3-(3-fluoro-phenyl)-[1,2,4]triazolo[4,3-b]pyridazin-6-yl)-2,2,N,N-tetramethyl-propane-1,3-diamine

2,2,N,N-tetramethyl-N-(3-phenyl-[1,2,4]triazolo[3,4-a]phthalazin-6-yl)-propane-1,3-diamine

N′-[3-(4-Methoxy-phenyl)-[1,2,4]triazolo[4,3-a ]phthalazin-6-yl)-2,2,N,N-tetramethyl-propane-1,3-diamine

6-(2-hydroxyethyl)oxy-3,7-diphenyl-[1,2,4]triazolo[4,3-b]pyridazine

6-(4-hydroxybutyl)oxy-3,7-diphenyl-[1,2,4]triazolo[4,3-b]pyridazine

2-(2-aminoprop-2-ylphenyl)-3-phenylquinazoline

or the pharmaceutically acceptable salt thereof.

Compounds within the scope of this invention which have been previously described as inhibitors of Akt but which have now been further identified by the instant assays as inhibitors of one or two of the Akt/PKB isoforms and are therefore useful in the present invention, and methods of synthesis thereof, can be found in the following patents, pending applications and publications, which are herein incorporated by reference:

All patents, publications and pending patent applications identified are hereby incorporated by reference.

The compounds used in the present method may have asymmetric centers and occur as racemates, racemic mixtures, and as individual diastereomers, with all possible isomers, including optical isomers, being included in the present invention. Unless otherwise specified, named amino acids are understood to have the natural “L” stereoconfiguration

The pharmaceutically acceptable salts of the compounds of this invention can be synthesized from the compounds of this invention which contain a basic moiety by conventional chemical methods. Generally, the salts are prepared by reacting the free base with stoichiometric amounts or with an excess of the desired salt-forming inorganic or organic acid in a suitable solvent or various combinations of solvents.

Abbreviations used in the description of the chemistry and in the Examples that follow are:

| | |
|---|---|
| Ac₂O | Acetic anhydride; |
| Boc | t-Butoxycarbonyl; |
| DBU | 1,8-diazabicyclo[5.4.0]undec-7-ene; |
| TFA: | trifluoroacetic acid |
| AA: | acetic acid |
| 4-Hyp | 4-hydroxyproline |
| Boc/BOC | t-Butoxycarbonyl; |
| Chg | cyclohexylglycine |
| DMA | dimethylacetamide |
| DMF | Dimethylformamide; |
| DMSO | dimethyl sulfoxide; |
| EDC | 1-(3-dimethylaminopropyl)-3-ethyl-carbodiimide |
| | hydrochloride; |
| EtOAc | Ethyl acetate; |
| EtOH | Ethanol; |
| FAB | Fast atom bombardment; |
| HOAt | 1-Hydroxy-7-azabenzotriazole |
| HOBt | 1-Hydroxybenzotriazole hydrate; |
| HOPO | 2-hydroxypyridine-N-oxide |
| HPLC | High-performance liquid chromatography; |
| IPAc | isopropylacetate |
| MeOH | methanol |
| RPLC | Reverse Phase Liquid Chromatography |
| THF | Tetrahydrofuran. |

Reactions used to generate the compounds which are inhibitors of Akt activity and are therefore useful in the methods of treatment of this invention are shown in the Schemes 1-6, in addition to other standard manipulations such as ester hydrolysis, cleavage of protecting groups, etc., as may be known in the literature or exemplified in the experimental procedures. Substituents R and R^{a}, as shown in the Schemes, represent the substituents R¹ and R²; however their point of attachment to the ring is illustrative only and is not meant to be limiting.

These reactions may be employed in a linear sequence to provide the compounds of the invention or they may be used to synthesize fragments that are subsequently joined by the alkylation reactions described in the Schemes.

Synopsis of Schemes 1-6:

The requisite intermediates are in some cases commercially available, or can be prepared according to literature procedures. As illustrated in Reaction Scheme 1, a suitably substituted phenylmaleic anyhydride i is treated with hydrazine to form the dihydropyridazone dione ii. Subsequent oxidative chlorination and reaction with a suitably substituted benzoic hydrazide provide the 6-chloro triazolo[4,3-b]pyridazine iii. This intermediate can then be treated with a variety of alcohols and amines to provide the compound iv.

Reaction Scheme 2 illustrates preparation of compounds useful in the methods of the instant invention having a cycloalkyl substituent at the 7-position. While a cyclobutyl group is illustrated, the sequence of reactions is generally applicable to incorporation of a variety of unsubstituted or substituted cycloalkyl moieties. Thus, 3,6-dichloropyridazine is alkylated via silver catalyzed oxidative decarboxylation with cyclobutyl carboxylic acid to provide the cyclobutyl dicloropyridazine v, which then undergoes the reactions described above to provide the instant compound vi.

Reaction Scheme 3 illustrates the same reaction sequence used to prepare compounds of the Formula I

Reaction Scheme 4 illustrates an alternative preparation of the instant compounds (Tetrahedron Letters 41:781-784 (2000)).

Reaction Scheme 5 illustrates a synthetic method of preparing the compounds of the Formula IV hereinabove.

Reaction Scheme 6 illustrates a synthetic method of preparing the compounds of the Formula III hereinabove.

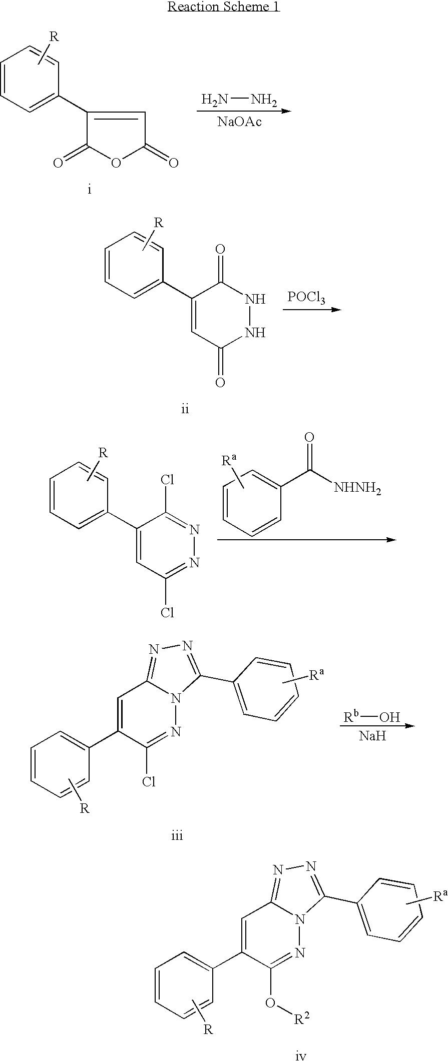

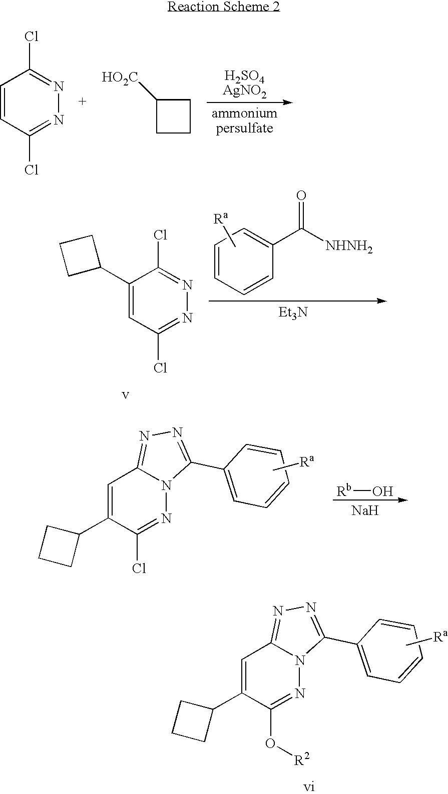

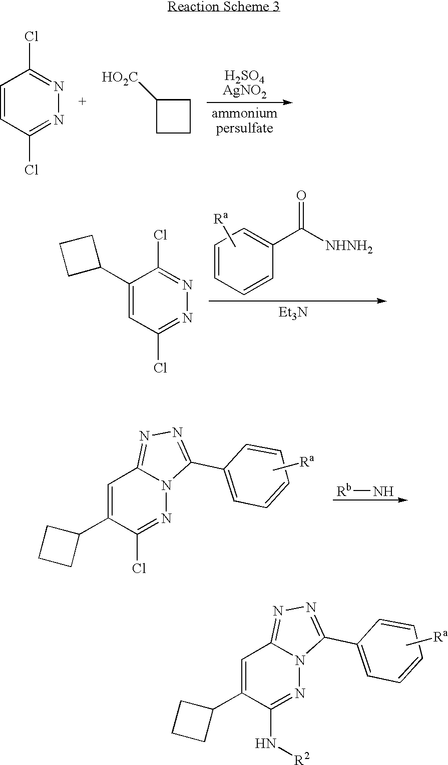

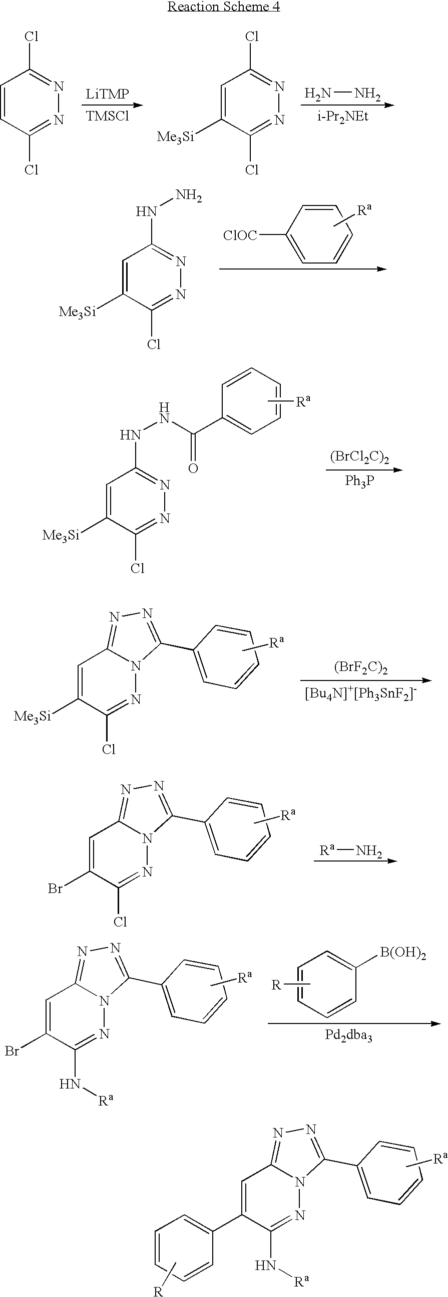

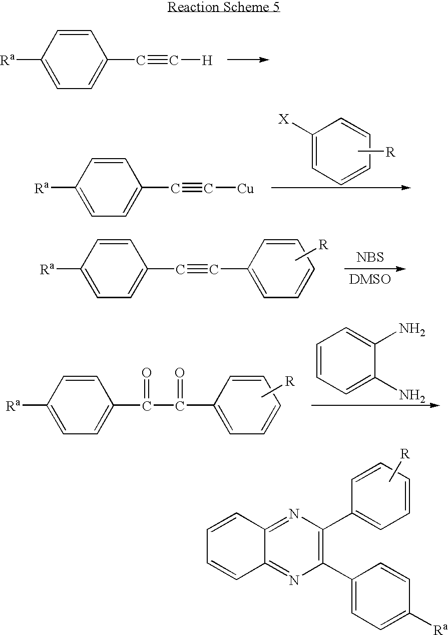

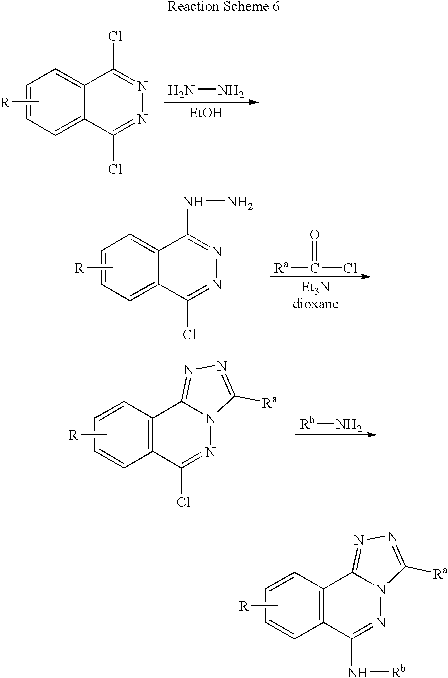

### EXAMPLES

Examples provided are intended to assist in a further understanding of the invention. Particular materials employed, species and conditions are intended to be further illustrative of the invention and not limitative of the reasonable scope thereof.

### Example 1

### N′-(7-Cyclobutyl-3-phenyl-[1,2,4]triazolo[4,3-b]pyridazin-6-yl)-2,2,N,N-tetramethyl-propane-1,3-diamine (Compound 1)

Step 1: 3,6-Dichloro-4-cyclobutyl pyridazine

Concentrated sulphuric acid (53.6 ml, 1.0 mol) was added carefully to a stirred suspension of 3,6-dichloropyridazine (50.0 g, 0.34 mol) in water (1.25 l). This mixture was then heated to 70° C. (internal temperature) before the addition of cyclobutane carboxylic acid (35.3 ml, 0.37 mol). A solution of silver nitrate (11.4 g, 0.07 mol) in water (20ml) was then added over approximately one minute. This caused the reaction mixture to become milky in appearance. A solution of ammonium persulphate (230 g, 1.0 mol) in water (0.63 l) was then added over 20-30 minutes. The internal temperature rose to approximately 85° C. During the addition the product formed as a sticky precipitate. Upon complete addition the reaction was stirred for an additional 5 minutes, then allowed to cool to room temperature. The mixture was then poured onto ice and basified with concentrated aqueous ammonia, with the addition of more ice as required to keep the temperature below 10° C. The aqueous phase was extracted with dichloromethane (×3). The combined extracts were dried (MgSO₄), filtered and evaporated to give the title compound (55.7 g, 82%) as an oil. ¹H nmr (CDCl₃) indicated contamination with approximately 5% of the 4,5-dicyclo-butyl compound. However, this material was used without further purification. Data for the title compound: ¹H NMR (360 MHz, d₆-DMSO) δ1.79-1.90 (1H, m), 2.00-2.09 (1H, m), 2.18-2.30 (2H, m), 2.33-2.40 (2H, m), 3.63-3.72 (1H, m), 7.95 (1H, s); MS (ES⁺) m/e 203 [MH]⁺, 205 [MH]⁺, 207 [MH]⁺.

Step) 2: 6-Chloro-7-cyclobutyl-3-phenyl-1,2,4-triazolo[4,3-b]pyridazine

A mixture of 3,6-dichloro-4-cyclobutylpyridazine from above (55.7 g, 0.27 mol), benzoic hydrazide (41.1 g, 0.30 mol) and triethylamine hydrochloride (41.5 g, 0.30 mol) in p-xylene (0.4 l) was stirred and heated at reflux under a stream of nitrogen for 24 hours. Upon cooling the volatiles were removed in vacuo. The residue was partitioned between dichloromethane and water. The aqueous layer was basified by the addition of solid potassium carbonate. Some dark insoluble material was removed by filtration at this stage. The aqueous phase was further extracted with dichloromethane (×2). The combined extracts were dried (MgSO₄), filtered and evaporated. The residue was purifiedby chromatography on silica gel eluting with 5%→10%→25% ethyl acetate/dichloromethane to give the title compound, (26.4 g, 34%) as an off-white solid. Data for the title compound: ¹H NMR (360 MHz, CDCl₃) δ1.90-2.00 (1H, m), 2.12-2.28 (3H, m), 2.48-2.57 (2H, m), 3.69-3.78 (1H, m), 7.49-7.59 (3H, m), 7.97 (1H, s), 8.45-8.48 (2H, m); MS(ES⁺) m/e 285 [MH]⁺, 287 [MH]⁺.

Step 3: N′-(7-Cyclobutyl-3-phenyl-[1,2,4]triazolo[4,3-b]pyridazin-6-yl)-2,2,N,N-tetramethyl-propane-1,3-diamine

6-Chloro-7-cyclobutyl-3-phenyl-[1,2,4]triazolo[4,3-b]pyridazine (100 mg) and N,N,2,2-tetramethyl-1,3-propanediamine (2 ml) were heated together in a sealed tube at 70° C. for 16 hours. Cooled and water (5 ml) added. Precipitate filtered, washed (water, ether) and dried. ¹H NMR (250 Mz, DMSO) δ1.20 (6H, s), 2.10 (1H, m), 2.24-2.65 (14H, m), 3.53-3.70 (2H, m), 7.69-7.82 (4H, m), 8.03 (1H, s), 8.70 (2H, m). MS (ES+) MH⁺=379

### Example 2

### N′-(7-Cyclobutyl-3-(3,5-difluoro-phenyl)-[1,2,4]triazolo[4,3-b]pyridazin-6-yl)-2,2,N,N-tetramethyl-propane-1,3-diamine (Compound 2)

The title compound was prepared in an analogous fashion to Example 1, except substituting 3,5-difluorobenzoic hydrazine for the benzoic hydrazine in Step 2. ¹H NMR.(360 MHz, CDCl₃) δ1.07 (6H, s), 1.99 (1H, m), 2.10-2.50 (13H, m), 3.31-3.35 (3H, m), 6.84-6.89 (1H, m), 7.63 (1H, s), 7.90 (1H, vbs), 8.20-8.23 (2H, m). MS (ES+) MH⁺=415

### Example 3

### N′-(7-Cyclobutyl-3-(3,4-difluoro-phenyl)-[1,2,4]triazolo[4,3-b]pyridazin-6-yl)-2,2,N,N-tetramethyl-propane-1,3-diamine (Compound 3)

The title compound was prepared in an analogous fashion to Example 1, except substituting 3,4-difluorobenzoic hydrazine for the benzoic hydrazine in Step 2. ¹H NMR (360 MHz, CDCl₃) δ1.07 (6H, s), 1.99-2.49 (14H, m), 3.30-3.33 (3H, m), 7.25-7.30 (1H, m), 7.62 (1H, s), 7.87 (1H, vbs), 8.32-8.34 (1H, m), 8.51-8.57 (1H, m). MS (ES+)MH⁺=415

### Example 4

### N′-(7-Cyclobutyl-3-(4-fluoro-phenyl)-[1,2,4]triazolo[4,3-b]pyridazin-6-yl)-2,2,N,N-tetramethyl-propane-1,3-diamine (Compound 4)

The title compound was prepared in an analogous fashion to Example 1, except substituting 4-fluorobenzoic hydrazine for the benzoic hydrazine in Step 2. ¹H NMR (360 MHz, CDCl₃) δ1.06 (6H, s), 1.98-2.49 (14H, m), 3.31-3.32 (3H, m), 7.18-7.26 (2H, m), 7.61 (1H, s), 7.80 (1H, vbs), 8.55-8.59 (2H, m). MS (ES+)MH⁺=397

### Example 5

### N′-(7-Cyclobutyl-3-(3-fluoro-phenyl)-[1,2,4]triazolo[4,3-b]pyridazin-6-yl)-2,2,N,N-tetramethyl-propane-1,3-diamine (Compound 5)

The title compound was prepared in an analogous fashion to Example 1, except substituting 3-fluorobenzoic hydrazine for the benzoic hydrazine in Step 2. ¹H NMR (360 MHz, CDCl₃) δ1.07 (6H, s), 1.96-2.50 (14H, m), 3.31-3.35 (3H, m), 7.10-7.15 (1H, m), 7.44-7.50 (1H, m), 7.63 (1H, m) 7.81 (1H, vbs), 8.35-8.42 (2H, m). MS (ES+)MH⁺=397

### Example 6

2,2,N,N-tetramethyl-N-(3-phenyl-[1,2,4]triazolo[3,4-a]phthalazin-6-yl)-propane-1,3-diamine (Compound 6)

Step 1: 1-Chloro-4-hydrazinophthalazine hydrochloride

To a stirred solution of hydrazine hydrate (40 ml) in ethanol (120 Ml) at 80° C. was added 1,4-dichlorophthalazine (20 g). This reaction mixture was stirred at 80° C. for 0.5 hours, then left to cool and the product was collected by filtration and dried under vacuum to give 1-chloro-4-hydrazinophthalazine hydrochloride (14.6 g). ¹H NMR (250 MHz, DMSO) 67 4.64 (2H, vbs), 7.2 (1H, vbs), 7.92 (4H, bm).

Step 2: 6-Chloro-3phenyl-1,2,4-triazolo[3,4-a]phthalazine

To a solution of 1-chloro-4-hydrazinophthalazine hydrochloride (10 g) in dioxan (220 ml) was added triethylamine (7.24 ml) and benzoyl chloride (6.04 ml). This mixture was heated at reflux for 8 hours under nitrogen. After cooling the reaction mixture was concentrated under vacuum and the solid obtained was collected by filtration, washed with water and diethyl ether and dried under vacuum, to yield the title compound (12.0 g). ¹H NMR (250 MHz, DMSO) δ7.60 (3H, m), 8.00 (1H, t, J=8.4 Hz), 8.19 (1H, t, J=8.4 Hz), 8.31 (3H, m), 8.61 (1H, d, J=6.3 Hz).

Step 3: 2,2,N,N-tetramethyl-N-(3-phenyl-[1,2,4]triazolo[3,4-a]phthalazin-6-yl)-propane-1,3-diamine

The title compound was prepared as described in Example 1, Step 3, but replacing the 6-Chloro-7-cyclobutyl-3-phenyl-[1,2,4]triazolo[4,3-b]pyridazine with the 6-Chloro-3phenyl-1,2,4-triazolo[3,4-a]phthalazine from Step 2. ¹H NMR (360 MHz, CDCl₃) δ1.13 (6H, s), 2.35 (2H, s), 2.46-2.50 (8H, m), 3.47 (2H, vbs), 7.16-7.27 (2H, m), 7.44-7.86 (5H, m), 8.55-8.57 (2H, m), 8.68 (1H, m). MS (ES+) MH⁺=375

### Example 7

### N′-[3-(4-Methoxy-phenyl)-[1,2,4]triazolo[4,3-a]phthalazin-6-yl)-2,2,N,N-tetramethyl-propane-1,3-diamine (Compound 7)

The title compound was prepared in an analogous fashion to Example 1, except substituting 3-fluorobenzoic hydrazine for the benzoic hydrazine in Step 2. ¹H NMR (360 MHz, CDCl₃) δ1.13 (6H, s), 2.45 (6H, s), 2.49 (2H, s), 3.45-3.46 (2H, m), 3.90 (3H, s) 7.04-7.07 (2H, m), 7.65-7.70 (2H, m), 7.80-7.84 (1H, m), 8.51 (2H, m), 8.66 (1H, m). MS (ES+)MH⁺=405

### Example 8

### 6-(2-Hydroxyethyl)oxy-3 7-diphenyl-[1,2,4]triazolo[4,3-b]pyridazine (Compound 8)

Step 1: 4-Phenyl-1,2-dihydropyridazine-3,6-dione

Phenylmaleic anhydride (30 g, 0.17 mol), sodium acetate trihydrate (28 g, 0.21 mol) and hydrazine monohydrate (10 ml, 0.21 mol) were heated together at reflux in 40% acetic acid (600 ml) for 18 hours. The mixture was cooled at 7° C. for 2 hours, then filtered. The solid was washed with diethyl ether and dried in vacuo to give 11 g (34%) of the title compound: ¹H NMR (250 MHz, DMSO-d₆) δ 7.16 (1H, br s), 7.44 (5H, m), 7.80 (2H, br s); MS (ES⁺) m/e 189 [MH⁺].

Step 2: 3,6 Dichloro-4-phenylpyridazine

4-Phenyl-1,2-dihydropyridazine-3,6-dinoe (3.4 g, 18 mmol) was heated at reflux in phosphorus oxychloride (70 ml) for 6 hours. The solution was concentrated in vacuo, then the residue was dissolved in dichloromethane (100 ml) and was neutralised by the addition of cold 10% aqueous sodium hydrogen carbonate (150 ml). The aqueous phase was washed with dichloromethane (2×50 ml), then the combined organic layers were washed with saturated aqueous sodium chloride (50 ml), dried (Na₂SO₄), and concentrated in vacuo to yield 3.9 g (97%) of the title compound: ¹H NMR (250 MHz, DMSO-d₆) δ7.54-7.66 (5H, m) 8.14 (1H, s); MS (ES⁺) m/e 225/227/229 [MH⁺].

Step 3: 6-Chloro-3,7-diphenyl-1,2,3-trizolo[4,3-b]pyridazine

3,6-Dichloro-4-phenylpyridazine (2.9 g, 13 mmol), benzoic hydrazide (1.9 g, 21 mmol) and triethylammonium chloride (2.0 g, 14 mmol) were heated together at reflux in xylene (150 ml) for three days. More benzoic hydrazide (0.88 g, 6.5 mmol) was added and the mixture was heated as before for another day. The solvent was removed in vacuo, and the residue was purified by flash chromatography (silica gel, 0-50% EtAOc/CH₂Cl₂) to afford 1.4 g (36%) of the title compound as a solid: ¹H NMR (250 MHz, CDCl₃) δ7.55 (8H, m), 8.12 (1H, s), 8.50 (2H, m); MS (ES⁺) m/e 307/309 [MH⁺].

Step 4: 6-(2-Hydroxyethyl)oxy-3,7-diphenyl-1,2,3-trizolo[4,3-b]pyridazine

Anhydrous DMF (1.5 ml) was added to a test tube containing NaH (13 mg) under nitrogen. Ethylene glycol (2 ml) was added and the mixture stirred at room temperature for 1 hour. The 6-chloro-3,7-diphenyl-1,2,3-trizolo[4,3-b]pyridazine (50 mg) (prepared as described in Step 3) was added as a solid and the reaction stirred at room temperature for 30 minutes and then heated at 60° C. for 8 hours and then stirred 10 hours at room temperature. The reaction mixture was then poured over 20 ml of hot water, the mixture cooled and the aqueous mixture extracted with ether. The organic phases were combined, washed with water, dried over MgSO₄, filtered and concentrated under vacuum to provide the title compound. ¹H NMR (CDCl₃, 500 MHz at 20° C.) δ8.48 (d, 2H, J =8.3), 8.04 (d, 1H, J =0.7), 7.61 (m, 2H), 7.57 (dd, 2H, J=7.6 and 8.1), 7.52 (m, 4H), 4.62 (dd, 2H, J =3.9 and 5.1), 4.04 (d, 2H, J=3.7). LC/MS (ES+) [M+1]=333.2.

### Example 9

### 6-(2-Hydroxybutyl)oxy-3,7-diphenyl-[1,2,4]triazolo[4,3-b]pyridazine (Compound 9)

The title compound was prepared by the procedure described in Example 1, but replacing ethylene glycol with 1,4-butanediol in Step 4. ¹H NMR (CDCl₃, 500 MHz at 20° C.) δ8.52 (dd, 2H, J=7.8 and 1.5), 8.02 (d, 1H, J=0.5), 7.58 (m, 4H), 7.51 (m, 4H), 4.53 (t, 2H, J =6.4), 3.69 (app. t, 2H, J =5.5), 1.97 (m 2H), 1.72 (m, 2H). LC/MS (ES+) [M+1]=361.3.

### Example 10

### Preparation of 2-(2-aminoprop-2-ylphenyl)-3-phenylquinazoline (Compound 10)

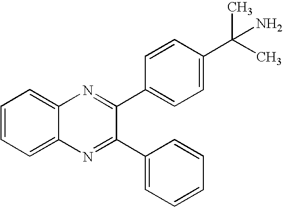

Step 1: Preparation of Ethyl 4-iodobenzoate

A mixture of 21.0 g of 4-iodobenzoic acid, 100 ml of absolute EtOH and 6 ml of concentrated sulfuric acid was refluxed with stirring for 6 days. At the end of this time the reaction mixture was concentrated by boiling and an additional 4 ml of concentrated sulfuric acid added. The mixture was then refluxed for an additional 11 days, after which the mixture was cooled and 50 g of ice and 150 ml Et₂O were added. The phases were separated and the, aqueous layer was extracted with Et₂O. The combined organic phases were washed with water, sat. aqueous NaHCO₃ and water. The organic phase was then dried over MgSO₄ and concentrated under vacuum to provide the title compound as a clear brownish liquid.

Step 2: Preparation of α,α-dimethyl-4-iodobenzyl alcohol

To a cooled (ice/H₂O) solution of 2.76 g of ethyl 4-iodobenzoate (prepared as described in Step 1) in 10 ml of anhyd. Et₂O was added, over a 5 minute period, 26.5 ml of 1.52M CH₃MgBr/ Et₂O solution. The mixture was stirred at ice bath temperature for 2.5 hours and then quenched by slow addition of 6 ml of H₂O. The reaction mixture was filtered and the solid residue rinsed with ether. The combined filtrates were dried over MgSO₄ and concentrated under vacuum to provide the title compound as a clear yellowish liquid.

Step 3: Preparation of α,α-dimethyl-4-iodo-N-formamido-benzyl amine

19 ml of glacial acetic acid was cooled in an ice bath until a slurry formed. 4.18 g of sodium cyanide was added over a 30 minute period. A cooled (ice/H₂O) solution of 10,3 ml conc. sulfuric acid in 95 ml glacial acetic acid was added to the cyanide solution over a 15 min. period. The ice bath was removed and 19.92 g of the α,α-dimethyl4-iodobenzyl alcohol (prepared as described in Step 2) was added over a 10 minute period. The resulting white suspension was stirred 90 minutes. And left standing overnight at room temperature. The reaction mixture was poured over ice and water and ether added. This mixture was neutralized with solid Na₂CO₃.

Step 4: Preparation of Copper (I) phenylacetylide

To a solution of 10.7 g of phenylacetylene in 500 ml of absolute ethanol was added a solution of 20 g of copper iodide in 250 ml of conc. NH₄OH and 100 ml of water. The solution was stirred 30 minutes and then filtered. The solid that was collected was washed with water, 95% aq. Ethanol and then ether. The solid was then collected and dried under vacuum to provide the title compound as a bright yellow solid.

Step 5: Preparation of 1-(2-formamidoprop-2-ylphenyl)-2-phenylacetylene

A mixture of 11.83 g of the iodophenyl compound described in Step 3, 6.74 g of Copper (I) phenylacetylide and 165 ml of dry pyridine was stirred at 120° C. for 72 hours. The reaction was then allowed to cool and the mixture was poured over approximately 300 g of ice and water with vigorous stirring. The mixture was then extracted with 1:1 benzene: diethylether. The organic solution was washed with 3N hydrochloric acid, dried over MgSO₄, filtered and concentrated to provide a solid, that was recrystallized from benzene/cyclohexane to provide the title compound.

Step 6: Preparation of 4-(2-formamidoprop-2-yl)-benzil

1-(2-formamidoprop-2-ylphenyl)-2-phenylacetylene from Step 5 (4.81 g) was dissolved in 30 ml of dried DMSO. N-Bromosuccinamide (NBS) (5.65 g) was added and the reaction stirred at room temperature for 96 hours. At this time 500 mg of NBS was added and the reaction stirred an additional 24 hours. The reaction mixture was then poured over water and the aqueous mixture extracted with benzene. The combined organic phases were washed with water and dried over MgSO₄. The organic slurry was then filtered and concentrated in vacuo to provide the title compound

Step 7: Preparation of 4-(2-aminoprop-2-yl)-benzil

4-(2-formamidoprop-2-yl)-benzil, prepared as described in Step 6 (6.17 g) was dissolved in 100 ml of glacial acetic acid, 84 ml of water and 6 ml of concentrated HCl. The mixture was stirred at reflux for 3 hours and then the solvent removed under vacuum at 60° C. The residue was converted to the free based form, extracted with organic solvent, washed with water, dried and concentrated to provide the title compound as an oil.

Step 8: Preparation of 2-(2-aminoprop-2-ylphenyl)-3-phenylquinazoline

A mixture of 1.0 g of 4-(2-aminoprop-2-yl)-benzil from Step 7, 0.406 g of o-phenylenediamine, 25 ml of glacial acetic acid and 15 ml of water was refluxed for 4.5 hours. The mixture was then allowed to stand overnight at room temperature. Most of the solvent was then removed under vacuum and the residue was taken up in 30 ml of water and 50 ml of 6 N aq. NaOH was added. The gum that precipitated was extracted with chloroform. The organic solution was washed with water, dried over MgSO₄ and concentrated under vacuum.

The residue was redissolved in chloroform and ethanolic HCl was added, precipitating out the hydrochloride salt. The salt was recrystallized from i-PrOH to provide the title compound as the hydrochloride salt—i-PrOH solvate (pale yellow plates). Mp 269° C.-271° C. (melted/resolidified at 250° C.).

Anal. Calc. for C₂₃H₂₁N₃.HCl.i-PrOH: C, 71.62; H, 6.94; N, 9.64. Found: C, 71.93; H, 6.97; N, 9.72 ¹H NMR (CDCl₃, 500 MHz at 20° C.) δ9.04 (broad s, 2.4H), 8.10 (d, 1H, J=7.8), 8.02 (d, 1H, J=7.8), 7.72 (dd, 1H, J=7.0 and 8.2), 7.66 (dd, 1H, J=7.0 and 8.2), 7.56 (m, 4H), 7.46 (dd, 2H, J=1.2 and 8.5), 7.31 (m, 3H), 1.81 (s, 6H). LC/MS (ES+) [M+1]=340.3.

### Example 11

### Preparation of 2,3-bis(4-aminophenyl)-quinoxaline (Compound 11)

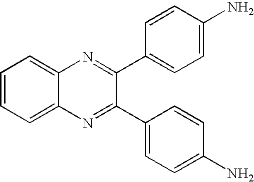

Step 1: Preparation of meso (d,1) hydrobenzoin

To a slurry of 97.0 g of benzil in 1 liter of 95% EtOH was added 20 g of sodium borohydride. After stirring 10 minutes, the mixture was diluted with 1 liter of water and the mixture was treated with activated carbon. The mixture was then filtered trough supercel and the filtrate heated and diluted with an additional 2 liters of water until it became slightly cloudy. The mixture was then cooled to 0 to 5° C. and the resulting crytals were collected and washed with cold water. The crystals were then dried in: vacuo.

Step 2: Preparation of 4,4′-dinitrobenzil

150 ml of fuming nitric acid was cooled to −10° C. and 25 g of the hydrobenzoin (prepared as described in Step 1) was added slowly portionwise while maintaining the temperature between −10° C. to −5° C. The reaction mixture was maintained at 0° C. for an additional 2 hours. 70 ml of water was added and the mixture was refluxed for 30 minutes and then poured onto 500 g of cracked ice. The residue was separated from the mixture by decantation and the residue was then boiled with 500 ml of water. The water layer was removed.

The remaining gum was dissolved in boiling acetone and the solution treated with decolorizing carbon and filtered. The filtrated was then cooled to −5° C. and the resulting crystals were collected and washed with cold acetone and dried in vacuo. An additional crop of crystalline title compound was obtained from recrystallization of the mother liquor residue.

Step 2: Preparation of 4,4′-diaminobenzil 3.8 g of 4,4′-dinitrobenzil was reduced under hydrogen with 3.8 g 10% Ru on C in EtOH. The mixture was filtered through Supracel and the filtrate concentrated under vacuum to dryness. The residue was dissolved in 50% denatured ethanol in water, treated with Darco and filtered. The filtrate was cooled to 0° C. and the resulting crystals were collected and washed with 50% denatured ethanol in water. The crystals were then dried under a heat lamp to give the title compound as a yellow powder.

Step 3: Preparation of 2,3-bis(4-aminophenyl)-quinoxaline

A mixture of 1.0 g (4.17 mmole) of 4,4′-diaminobenzil and 0.45 g of o-phenylenediamine in 250 ml glacial acetic acid was heated at 50° C. for 15 minutes, then stirred for 16 hours at room temperature. The mixture was then heated to 80° C. and allowed to cool slowly. The solvent was removed under vacuum and the residue was redissolved in ethanol and that was removed under vacuum.

The solid residue was recrystalized from boiling acetone, and the solid collected. The residue from the mother liquors was recrystalized form 95% EtOH and the resulting crystals combined with the crystals from the acetone crystalization and all were recrystalized from 1:1 abs. EtOH:95% EtOH to provide crystalline material. The crystals were dried for over 5 hours at 110° C. under vacuum to provide the title compound.

Anal. Calc. for C₂₀H₁₆N₄: C, 76.90; H, 5.16; N, 17.94. Found: C, 76.83; H, 4.88; N, 18.16 ¹H NMR (CDCl₃, 500 MHz at 20° C.) δ8.08 (m, 2H), 7.67 (m, 2H), 7.39 (m, 4H), 6.64 (m, 4H), 3.80 (broad s, 4H). LC/MS (ES+) [M+1]=313.3.

### Example 12

### Cloning of the human Akt isoforms and ΔPH-Akt1

The pS2neo vector (deposited in the ATCC on Apr. 3, 2001 as ATCC) was prepared as follows: The pRmHA3 vector (prepared as described in Nucl. Acid Res. 16:1043-1061 (1988)) was cut with BgII and a 2734 bp fragment was isolated. The pUChsneo vector (prepared as described in EMBO J. 4:167-171 (1985)) was also cut with BgII and a 4029 bp band was isolated. These two isolated fragments were ligated together to generate a vector termed pS2neo-1. This plasmid contains a poly-linker between a metallothionine promoter and an alcohol dehydrogenase poly A addition site. It also has a neo resistance gene driven by a heat shock promoter. The pS2neo-1 vector was cut with Psp5II and BsiWI. Two complementary oligonucleotides were synthesized and then annealed (CTGCGGCCGC (SEQ.ID.NO.: 1) and GTACGCGGCCGCAG (SEQ.ID.NO.: 2)). The cut pS2neo-1 and the annealed oligonucleotides were ligated together to generate a second vector, pS2neo. Added in this conversion was a NotI site to aid in the linearization prior to transfection into S2 cells.

Human Akt1 gene was amplified by PCR (Clontech) out of a human spleen cDNA (Clontech) using the 5′ primer: 5′ CGCGAATTCAGATCTAC CASTEAGCGACGTGGCTATTGTG3′ (SEQ.ID.NO.: 3), and the 3′ primer: 5′CGCTCTAGAGGATCCTCAGGCCGTGCTGCTGGC3′ (SEQ.ID.NO.: 4). The 5′ primer included an EcoRI and BglII site. The 3′ primer included an XbaI and BamHI site for cloning purposes. The resultant PCR product was subcloned into pGEM3Z (Promega) as an EcoRI/Xba I fragment. For expression/purification purposes, a middle T tag was added to the 5′ end of the full length Akt1 gene using the PCR primer: 5′GTACGATGCTGAACGATATCTTCG 3′ (SEQ.ID.NO.: 5). The resulting PCR product encompassed a 5′ KpnI site and a 3′ BamHI site which were used to subclone the fragment in frame with a biotin tag containing insect cell expression vector, pS2neo.

For the expression of a pleckstrin homology domain (PH ) deleted (Δ aa 4-129, which includes deletion of a portion of the Akt1 hinge region) version of Akt1, PCR deletion mutagenesis was done using the full length Akt1 gene in the pS2neo vector as template. The PCR was carried out in 2 steps using overlapping internal primers: (5′GAATACATGCCGATGGAAAGCGACAGGGGCTGAAGAG ATGGAGGTG 3′ (SEQ.ID.NO.: 6), and 5′CCCCTCCATCTCTTCAGCCCCΔGTC GCTTTCCATCGGCATGTATTC 3′ (SEQ.ID.NO.: 7)) which encompassed the deletion and 5′ and 3′ flanking primers which encompassed the KpnI site and middle T tag on the 5′ end. The final PCR product was digested with KpnI and SmaI and ligated into the pS2neo full length Akt1 KpnI/Sma I cut vector, effectively replacing the 5′ end of the clone with the deleted version.

Human Akt3 gene was amplified by PCR of adult brain cDNA (Clontech) using the amino terminal oligo primer: 5′ GAATTCAGATCTACCATGA GCGATGTTACCATTGTG 3′ (SEQ.ID.NO.: 8); and the carboxy terminal oligo primer: 5′ TCTAGATCTTATTCTCGTCCACTTGCAGAG 3′ (SEQ.ID.NO.: 9). These primers included a 5′ EcoRI/BglII site and a 3′ XbaI/BglII site for cloning purposes. The resultant PCR product was cloned into the EcoRI and XbaI sites of pGEM4Z (Promega). For expression/purification purposes, a middle T tag was added to the 5′ end of the full length Akt3 clone using the PCR primer: 5′ GGTACC ATGGAATACATGCCGATGGAAAGCGATGTTACCATTGTGAAG 3′ (SEQ.ID. NO.: 10). The resultant PCR product encompassed a 5′ KpnI site which allowed in frame cloning with the biotin tag containing insect cell expression vector, pS2neo.

Human Akt2 gene was amplified by PCR from human thymus cDNA (Clontech) using the amino terminal oligo primer: 5′ AAGCTTAGATCTACCATGA ATGAGGTGTCTGTC 3′ (SEQ.ID.NO.: 11); and the carboxy terminal oligo primer: 5′ GAATTCGGATCCTCACTCGCGGATGCT GGC 3′ (SEQ.ID.NO.: 12). These. primers included a 5′ Hind/BglII site and a 3′ EcoRI/BamHI site for cloning purposes. The resultant PCR product was subcloned into the HindIII/EcoRI sites of pGem3Z (Promega). For expression/purification purposes, a middle T tag was added to the 5′ end of the full length Akt2 using the PCR primer: 5′ GGTACCATGG AATACATGCCGATGGAAAATGAGGTGTCTGTCATCAAAG 3 ′ (SEQ.ID.NO.: 13). The resultant PCR product was subcloned into the pS2neo vector as described above.

### Example 13

### Expression of human Akt isoforms and ΔPH-Akt1

The DNA containing the cloned Akt1, Akt2, Akt3 and ΔPH-Akt1 genes in the pS2neo expression vector was purified and used to transfect Drosophila S2 cells (ATCC) by the calcium phosphate method. Pools of antibiotic (G418, 500 μg/ml) resistant cells were selected. Cell were expanded to a 1.0L volume (˜7.0×10⁶/ml), biotin and CuSO₄ were added to a final concentration of 50 μM and 50 mM respectively. Cells were grown for 72 hours at 27° C. and harvested by centrifugation. The cell paste was frozen at −70° C. until needed.

### Example 14

### Purification of Human Akt Isoforms and ΔPH-Akt1

Cell paste from one liter of S2 cells, described in Example 13, was lysed by sonication with 50 mls 1% CHAPS in buffer A: (50 mM Tris pH 7.4, 1 mM EDTA, 1 mM EGTA, 0.2 mM AEBSF, 10 μg/ml benzamidine, 5 μg/ml of leupeptin, aprotinin and pepstatin each, 10% glycerol and 1 mM DTT). The soluble fraction was purified on a Protein G Sepharose fast flow (Pharmacia) column loaded with 9 mg/ml anti-middle T monoclonal antibody and eluted with 75 μM EYMPME (SEQ.ID.NO.: 14) peptide in buffer A containing 25% glycerol. Akt/PKB containing fractions were pooled and the protein purity evaluated by SDS-PAGE. The purified protein was quantitated using a standard Bradford protocol. Purified protein was flash frozen on liquid nitrogen and stored at −70° C.

### Example 15

### Kinase Assays

This procedure describes a kinase assay which measures phosphorylation of a biotinylated GSK3-derived peptide by human recombinant active Akt/PBK isoforms or Akt/PBK mutants. The ³³P-labeled biotinylated product can be captured and detected using Streptavidin coated Flashplates (NEN LifeSciences) or Streptavidin Membrane Filter Plates (Promega). Alternatively, a GSK3-derived peptide with 2 added lysine residues was used as the substrate and subsequently captured using Phosphocellulose Membrane Filter Plates (Polyfiltronics).

Materials:

Active human Akt: The following active human Akt isoforms were utilized in the in vitro assays: active human Akt1 (obtained from Upstate Biotechnology, catalog no. 14-276, 15 μg/ 37 μl (6.76 μM)) or recombinant lipid activated Akt1 (prepared as described in Example 14); Akt2 (prepared as described in Example 14); Akt3 (prepared as described in Example 14); and delta PH-Akt1 (prepared as described in Example 14).

Akt Specific Peptide Substrate:

GSK3α (S21) Peptide #3928, biotin-GGRARTSSFAE PG (SEQ.ID.NO.: 15), FW=1517.8 (obtained from Macromolecular Resources) for Streptavidin Flashplate or Streptavidin Filter Plate detection.

GSK3α (S21) Peptide #G80613, KKGGRARTSSFAEPG (SEQ.ID.NO.: 16), FW=1547.8 (obtained from Research Genetics) for Phosphocellulose filter plate detection.

Standard Assay Solutions:

A. 10× AADKA Assay Buffer:

500 mM HEPES, pH 7.5

1% PEG

1 mM EDTA

1 mM EGTA

20 mM β-Glycerol phosphate

B. Active Akt (500 nM): Diluent (1× Assay buffer, 10% glycerol, 0.1% β-mercaptoethanol, 1.0 μM microcystin LR and 1.0 mM EDTA) was added to a vial containing 37 μl of active Akt isoform (6.76 μM). Aliquots were flash frozen in liquid N₂ and stored at −70° C.

C. 1 mM Akt specific peptide substrate in 50 mM Tris pH 7.5, 1 mM DTT.

D. 100 mM DTT in di H₂O.

E. 100× Protease Inhibitor Cocktail (PIC): 1 mg/ml benzamidine, 0.5 mg/ml pepstatin, 0.5 mg/ml leupeptin, 0.5 mg/mil aprotinin.

F. 3 mM ATP, 200 mM MgCl₂ in H₂O, pH 7.9.

G. 50% (v/v) Glycerol.

H. 1% (wt/v) BSA (10 mg/ml) in diH20, 0.02% (w/v) NaN₃.

I. 125 mM EDTA.

J. 0.75% (wt/v) Phosphoric Acid.

K. 2.5 M Potassium Chloride,

L. Tris Buffered Saline (TBS), 25 mM Tris, 0.15 M Sodium Chloride, pH 7.2 (BupH Tris Buffered Saline Pack, Pierce catalog no. 28376).

Procedure for Streptavidin Flash Plate Assay:

Step, 1:

A 1 μl solution of the test compound in 100% DMSO was added to 20 μl of 2× substrate solution (20 μM GSK3 Peptide, 300 μM ATP, 20 mM MgCl₂, 20 μCi/ml [γ³³P] ATP, 1× Assay Buffer, 5% glycerol, 1 mM DTT, 1× PIC, 0.1% BSA and 100 mM KCl). Phosphorylation reactions were initiated by adding 19 μl of 2× Enzyme solution (6.4 nM active Akt/PKB, 1× Assay Buffer, 5% glycerol, 1 mM DTT, 1× PIC and 0.1% BSA). The reactions were then incubated at room temperature for 45 minutes.

Step 2:

The reaction was stopped by adding 170 μl of 125 mM EDTA. 200 μl of stopped reaction was transferred to a Streptavidin Flashplate® PLUS (NEN Life Sciences, catalog no. SMP103). The plate was incubated for ≧10 minutes at room temperature on a plate shaker. The contents of each well was aspirated, and the wells rinsed 2 times with 200 μl TBS per well. The wells were then washed 3 times for 5 minutes with 200 μl TBS per well with the plates incubated at room temperature on a platform shaker during wash steps.

The plates were covered with sealing tape and counted using the Packard TopCount with the appropriate settings for counting [³³P] in Flashplates.

Procedure for Streptavidin Filter Plate Assay:

The enzymatic reactions as described in Step I of the Streptavidin Flash Plate Assay above were performed.

Step 2:

The reaction was stopped by adding 20 μl of 7.5M Guanidine Hydrochloride. 50 μl of the stopped reaction was transferred to the Streptavidin filter plate (SAM^{2TM} Biotin Capture Plate, Promega, catalog no. V7542) and the reaction was incubated on the filter for 1-2 minutes before applying vacuum.

The plate was then washed using a vacuum manifold as follows: 1) 4×200 μl/well of 2M NaCl; 2) 6×200 μl/well of 2M NaCl with 1% H₃PO₄; 3)2×200 μl/well of diH₂0; and 4)2×100 μl/well of 95% Ethanol. The membranes were then allowed to air dry completely before adding scintillant.

The bottom of the plate was sealed with white backing tape, 30 μl/well of Microscint 20 (Packard Instruments, catalog no. 6013621) was added. The top of the plate was sealed with clear sealing tape, and the plate then counted using the Packard TopCount with the appropriate settings for [³³P] with liquid scintillant.

Procedure for Phosphocellulose Filter Plate Assay:

Step 1:

The enzymatic reactions were performed as described in Step 1 of the Streptavidin Flash Plate Assay (above) utilizing KKGGRARTSSFAEPG (SEQ.ID. NO.: 16) as the substrate in place of biotin-GGRARTSSFAEPG.

Step 2:

The reaction was stopped by adding 20 μl of 0.75% H₃PO₄. 50 μl of stopped reaction was transferred to the filter plate (UNIFILTER™, Whatman P81 Strong Cation Exchanger, White Polystyrene 96 Well Plates, Polyfiltronics, catalog no. 7700-3312) and the reaction incubated on the filter for 1-2 minutes before applying vacuum.

The plate was then washed using a vacuum manifold as follows: 1) 9×200 μl/well of 0.75% H₃PO₄; and 2) 2×200 μl/well of diH₂0. The bottom of the plate was sealed with white backing tape, then 30 μl/well of Microscint 20 was added. The top of the plate was sealed with clear sealing tape, and the plate counted using the Packard TopCount with the appropriate settings for [³³P] and liquid scintillant.

PKA Assay

Each individual PKA assay consists of the following components:

1) 10 μl 5× PKA assay buffer (200 mM Tris pH7.5, 100 mM MgCl₂, 5 mM 2-mercaptoethanol, 0.5 mM EDTA)

2) 10 μl of a 50 μM stock of Kemptide (Sigma) diluted into water

3) 10 μl ³³P-ATP (prepared by diluting 1.0 μl ³³P-ATP [10 mCi/ml] into 200 μl of a 50 μM stock of unlabeled ATP)

4) 10 μl appropriate solvent control dilution or inhibitor dilution

5) 10 μl of a 70 nM stock of PKA catalytic subunit (UBI catalog #14-114) diluted in 0.5 mg/ml BSA

The final assay concentrations were 40 mM Tris pH 7.5, 20 mM MgCl₂, 1 mM 2-mercaptoethanol, 0.1 mM EDTA, 10 μM Kemptide, 10 μM ³³P-ATP, 14 nM PKA and 0.1 mg/ml BSA.

Assays were assembled in 96 deep-well assay plates. Components #3 and #4 were premixed and in a separate tube, a mixture containing equal volumes of components #1, #2, and #5 was prepared. The assay reaction was initiated by adding 30 μl of the components #1, #2, and #5 mixture to wells containing ³³P-ATP and inhibitor. The liquid in the assay wells was mixed and the assay reactions incubated for 20 minutes at room temperature. The reactions were stopped by adding 50 μl 100 mM EDTA and 100 mM sodium pyrophosphate and mixing.

The enzyme reaction product (phosphorylated Kemptide) was quantitated using p81 phosphocellulose 96 well filter plates (Millipore). Each well of a p81 filter plate was fill with 75 mM phosphoric acid. The wells were aspirated and 170 μl of 75 mM phosphoric acid was added to each well. A 3040 III aliquot from each stopped PKA reaction was added to corresponding wells on the filter plate contained the phosphoric acid. The peptide was trapped on filter following the application of a vacuum. The filters were washed 5× by filling wells with 75 mM phosphoric acid followed by aspiration. After the final wash, the filters were allowed to air dry. 30 μl scintillation fluid was added to each well and the filters counted on a TopCount (Packard).

PKC Assay

Each PKC assay consists of the following components:

1) 5 μl 10×PKC co-activation buffer (2.5 mM EGTA, 4 mM CaCl₂)

2) 10 μl 5×PKC activation buffer (1.6 mg/ml phosphatidylserine, 0.16 mg/ml diacylglycerol, 100 mM Tris pH 7.5, 50 mM MgCl, 5 mM 2-mercaptoethanol)

3) 5 μl ³³P-ATP (prepared by diluting 1.0 μl ³³P-ATP [10 mCi/nil] into 100 μl of a 100 μM stock of unlabeled ATP)

4) 10 μl of a 350 μg/ml stock of myelin basic protein (MBP, UBI) diluted in water

5) 10 μl appropriate solvent control or inhibitor dilution

6) 10 μl of a 50 ng/ml stock of PKC (mix of isoforms from UBI catalog #14-115) diluted into 0.5 mg/ml BSA

Final assay concentrations were as follows: 0.25 mM EGTA, 0.4 mM CaCl, 20 mM Tris pH 7.5, 10 mM MgCl, 1 mM 2-mercaptoethanol, 0.32 mg/ml phosphatidylserine, 0.032 mg/mil diacylglycerol, 10 μM 33P-ATP, 70 μg/ml MBP, 10 ng/ml PKC, 0.1 mg/ml BSA.

Assays are performed using 96 deep well assay plates. In each assay well 10 μl of solvent control or appropriate inhibitor dilution with 5 μl ³³P-ATP (components #5 and #3) were premixed. In a separate tube, a mixture containing equal volumes of components #1, #2, #4, and #6 was prepared. The assay reaction was initiated by adding 35 μl of the components #1, #2, #4, and #6 mixture to wells containing ³³P-ATP and inhibitor. The liquid in the assay wells was thoroughly mixed and the assay reactions incubated for 20 minutes at room temperature. The reactions were stopped by adding 100 mM EDTA (50 μl) and 100 mM sodium pyrophosphate (50 μl) and mixing. Phosphorylated MBP was collected on PVDF membranes in 96 well filter plates and quantitated by scintillation counting.

The results from testing the compounds described in Examples 1-11 in the assays described above are shown in Table 1:

TABLE 1

| GSK3 Peptide Substrate | | | | Counter | |
|---|---|---|---|---|---|
| IC₅₀ (μM) | | | | screens | |
| | Akt-1 delta | | | IC₅₀ (μM) | |
| Akt-1 | PH | Akt2 | Akt3 | PKA | PKC |

| | | | | | | | | | |
|---|---|---|---|---|---|---|---|---|---|
| Compound 1 | 1.4 (5) | >50 | (2) | >50 | (2) | >50 | (2) | >40 | >40 |
| Compound 2 | 0.42 | >50 | | >50 | | >50 | | >40 | >40 |
| Compound 3 | 0.91 | >50 | | >50 | | >50 | | >40 | >40 |
| Compound 4 | 2.03 | >50 | | >50 | | >50 | | >40 | >40 |
| Compound 5 | 0.4 | >50 | | >50 | | >50 | | >40 | >40 |
| Compound 7 | 3.88 | >50 | | >50 | | >50 | | >40 | >40 |
| Compound 6 | 10.5 | >50 | | >50 | | >50 | | >40 | >40 |
| Compound 8 | 15.9 | >50 | | >50 | | >50 | | >40 | >40 |
| Compound 9 | 4.65 | >50 | | >50 | | >50 | | >40 | >40 |
| Compound | 1.68 | >50 | | 12.5 | | >50 | | >80 | >80 |
| 10 | | | | | | | | | |
| Compound | 6.1 (4) | >50 | | 45 | | >100 | | >80 | >80 |
| 11 | | | | | | | | | |

### Example 16

### Cell Based Assays to Determine Inhibition of Akt/PKB

Cells (for example LnCaP or a PTEN^{(-/-)} tumor cell line with activated Akt/PKB) were plated in 100 mM dishes. When the cells were approximately 70 to 80% confluent, the cells were refed with 5 mls of fresh media and the test compound added in solution. Controls included untreated cells, vehicle treated cells and cells treated with either LY294002 (Sigma) or wortmanin ( Sigma ) at20 μM or 200 nM, respectively. The cells were incubated for 2 hours, and the media removed, The cells were washed with PBS, scraped and transferred to a centrifuge tube. They were pelleted and washed again with PBS. Finally, the cell pellet was resuspended in lysis buffer (20 mM Tris pH8, 140 mM NaCl, 2 mM EDTA, 1% Triton, 1 mM Na Pyrophosphate, 10 mM β-Glycerol Phosphate, 10 mM NaF, 0.5 mm NaVO₄, 1 μM Microsystine, and 1×Protease Inhibitor Cocktail), placed on ice for 15 minutes and gently vortexed to lyse the cells. The lysate was spun in a Beckman tabletop ultra centrifuge at 100,000×g at 4° C. for 20 minutes. The supernatant protein was quantitated by a standard Bradford protocol (BioRad) and stored at −70° C. until needed.

Proteins were immunoprecipitated (IP) from cleared lysates as follows: For Akt1 /PKBα, lysates are mixed with Santa Cruz sc-7126 (D-17) in NETN (100 mM NaCl, 20 mM Tris pH 8.0, 1 mM EDTA, 0.5% NP-40) and Protein A/G Agarose (Santa Cruz sc-2003) was added. For Akt2/PKBβ, lysates were mixed in NETN with anti-Akt-2 agarose (Upstate Biotechnology #16-174) and for Akt3/PKBγ, lysates were mixed in NETN with anti-Akt-3 agarose (Upstate Biotechnology #16-175). The IPs were incubated overnight at 4° C., washed and seperated by SDS-PAGE.

Western blots were used to analyze total Akt, pThr308 Akt, pSer473 Akt, and downstream targets of Akt using specific antibodies (Cell Signaling Technology): Anti-Total Akt (cat. no. 9272), Anti-Phopho Akt Serine 473 (cat. no. 9271), and Anti-Phospho Akt Threonine 308 (cat. no. 9275). After incubating with the appropriate primary antibody diluted in PBS+0.5% non-fat dry milk (NFDM) at 4° C. overnight, blots were washed, incubated with Horseradish peroxidase (HRP)-tagged secondary antibody in PBS+0.5% NFDM for 1 hour at room temperature. Proteins were detected with ECL Reagents (Amersham/Pharmacia Biotech RPN2134).

### Example 17

### Heregulin Stimulated Akt Activation

MCF7 cells (a human breast cancer line that is PTEN^{+/+}) were plated at 1×10⁶ cells per 100 mM plate. When the cells were 70-80% confluent, they were re-fed with 5 ml of serum free media and incubated overnight. The following morning, compound was added and the cells were incubated for 1-2 hours, heregulin was added (to induce the activation of Akt) for 30 minutes and the cells were analyzed as described above.

### Example 18

### Inhibition Of Tumor Growth

In vivo efficacy as an inhibitor of the growth of cancer cells may be confirmed by several protocols well known in the art.

Human tumor cells from cell lines which exhibit a deregulation of the PI3K pathway (such as LnCaP, PC3, C33a, OVCAR-3, MDA-MB-468 or the like) are injected subcutaneously into the left flank of 8-12 week old female nude mice (Harlan) on day 0. The mice are randomly assigned to a vehicle, compound or combination treatment group. Daily subcutaneous administration begins on day 1 and continues for the duration of the experiment. Alternatively, the inhibitor test compound may be administered by a continuous infusion pump. Compound, compound combination or vehicle is delivered in a total volume of 0.1 ml. Tumors are excised and weighed when all of the vehicle-treated animals exhibited lesions of 0.5-1.0 cm in diameter, typically 4 to 5.5 weeks after the cells were injected. The average weight of the tumors in each treatment group for each cell line is calculated.

What is claimed is:

1. A method for treating cancer in a mammal in need thereof which comprises administering to said mammal amounts of a selective inhibitor of the activity of one or more of the isoforms of Akt.

2. The method according to claim 1 wherein the selective inhibitor inhibits the phosphorylation of one or more of the isoforms of Akt by upstream kinases and inhibits the phosphorylation of protein targets of an isoform or isoforms of Akt by the activated isoform or isoforms of Akt.

3. The method according to claim 1 wherein the inhibitor is a selective inhibitor of the activity of Akt 1.

4. The method according to claim 1 wherein the inhibitor is a selective inhibitor of the activity of Akt 2.

5. The method according to claim 1 wherein the inhibitor is a selective inhibitor of the activity of Akt 1 and Akt 2.

6. The method according to claim 2 wherein the inhibitor is a selective inhibitor of the activity of Akt 3.

7. A method for treating cancer in a mammal in need thereof which comprises administering to said mammal amounts of an inhibitor of the activity of one or more of the isoforms of Akt wherein the inhibition by the inhibitor is dependent on the presence of the pleckstrin homology domain of the isoforms of Akt.

8. The method according to claim 7 wherein the inhibitor is a selective inhibitor of the activity of Akt 1.

9. The method according to claim 7 wherein the inhibitor is a selective inhibitor of the activity of Akt 2.

10. The method according to claim 7 wherein the inhibitor is a selective inhibitor of the activity of Akt 3.

11. The method according to claim 7 wherein the inhibitor is a selective inhibitor of Akt-1 and Akt-2.

12. The method according to claim 7 wherein the inhibitor is a selective inhibitor of Akt-1, Akt-2 and Akt-3.

13. A method for treating cancer in a mammal in need thereof which comprises administering to said mammal amounts of an inhibitor of the activity of one or more of the isoforms of Akt wherein the inhibition by the inhibitor is dependent on the presence of the hinge region of the isoforms of Akt.

14. The method according to claim 3 wherein the inhibitor is a selective inhibitor of the activity of Akt 1.

15. The method according to claim 13 wherein the inhibitor is a selective inhibitor of the activity of Akt 2.

16. The method according to claim 13 wherein the inhibitor is a selective inhibitor of the activity of Akt 3.

17. The method according to claim 13 wherein the inhibitor is a selective inhibitor of Akt-1 and Akt-2.

18. The method according to claim 13 wherein the inhibitor is a selective inhibitor of Akt-1, Akt-2 and Akt-3.

19. A method for treating cancer in a mammal in need thereof which comprises administering to said mammal amounts of an inhibitor of the activity of one or more of the isoforms of Akt wherein the inhibition by the inhibitor is dependent on the presence of the pleckstrin homology domain and the hinge region of the isoforms of Akt.

20. The method according to claim 19 wherein the inhibitor is a selective inhibitor of the activity of Akt 1.

21. The method according to claim 19 wherein the inhibitor is a selective inhibitor of the activity of Akt 2.

22. The method according to claim 19 wherein the inhibitor is a selective inhibitor of the activity of Akt 3.

23. The method according to claim 19 wherein the inhibitor is a selective inhibitor of Akt-1 and Akt-2.

24. The method according to claim 19 wherein the inhibitor is a selective inhibitor of Akt-1, Akt-2 and Akt-3.

25. The method according to claim 1 wherein the inhibitor is a selective inhibitor of the activity of Akt-1, but is not an inhibitor of the activity of a modified Akt-1 that lacks the pleckstrin homology domain.

26. The method according to claim 1 wherein the inhibitor is a selective inhibitor of the activity of Akt-2, but is not an inhibitor of the activity of a modified Akt-2 that lacks the pleckstrin homology domain.

27. The method according to claim 1 wherein the inhibitor is a selective inhibitor of the activity of Akt-3, but is not an inhibitor of the activity of a modified Akt-3 that lacks the pleckstrin homology domain.

28. The method according to claim 1 wherein the inhibitor is a selective inhibitor of the activity of Akt-1 and Akt-2, but is not an inhibitor of the activity of a modified Akt-1 that lacks the pleckstrin homology domain, a modified Akt-2 that lacks the pleckstrin homology domain or both a modified Akt-1 and a modified Akt-2 protein that lack their pleckstrin homology domains.

29. The method according to claim 1 wherein the inhibitor is a selective inhibitor of the activity of Akt-1, Akt-2 and Akt-3, but is not an inhibitor of the activity of a modified Akt-1 that lacks the pleckstrin homology domain, a modified Akt-2 that lacks the pleckstrin homology domain, a modified Akt-3 that lacks the pleckstrin homology domain or two or three modified Akt isoforms that lack their pleckstrin homology domains.

30. A method for identifying a compound that is a selective inhibitor of one, two or three of the Akt isoforms, whose inhibitory efficacy is dependent on the pleckstrin homology domain, that comprises the steps of:
a) determining the efficacy of a test compound in inhibiting the activity of an Akt isoform;
b) determining the efficacy of the test compound in inhibiting the activity of the Akt isoform that has been modified to delete the pleckstrin homology domain; and
c) comparing the activity of the test compound against the Akt isoform with the activity of the test compound against the modified Akt isoform lacking the pleckstrin homology domain.

31. A method for identifying a compound that is a selective inhibitor of one, two or three of the Akt isoforms, whose inhibitory efficacy is dependent on the hinge region of Akt, that comprises the steps of:
a) determining the efficacy of a test compound in inhibiting the activity of an Akt isoform;
b) determining the efficacy of the test compound in inhibiting the activity of the Akt isoform that has been modified to delete the pleckstrin homology domain;
c) determining the efficacy of the test compound in inhibiting the activity of the Akt isoform that has been modified to delete the pleckstrin homology domain and the hinge region; and
d) comparing the activity of the test compound against the Akt isoform, the activity of the test compound against the modified Akt isoform lacking the PH domain, and the activity of the test compound against the modified Akt isoform lacking the pleckstrin homology domain and the hinge region.

32. A modified Akt isoform lacking only the pleckstrin homology domain.

33. A modified Akt isoform lacking only the hinge region.

34. A modified Akt isoform lacking the full pleckstrin homology domain and the full hinge region.


SEQUENCE LISTING (16 sequences)

SEQ ID NO: 1 - DNA, 10 bp, Artificial Sequence
  Completely synthetic DNA Sequence
ctgcggccgc

SEQ ID NO: 2 - DNA, 14 bp, Artificial Sequence
  Completely synthetic DNA Sequence
gtacgcggccgcag

SEQ ID NO: 3 - DNA, 39 bp, Artificial Sequence
  Completely synthetic DNA Sequence
cgcgaattcagatctaccatgagcgacgtggctattgtg

SEQ ID NO: 4 - DNA, 33 bp, Artificial Sequence
  Completely synthetic DNA Sequence
cgctctagaggatcctcaggccgtgctgctggc

SEQ ID NO: 5 - DNA, 24 bp, Artificial Sequence
  Completely synthetic DNA Sequence
gtacgatgctgaacgatatcttcg

SEQ ID NO: 6 - DNA, 45 bp, Artificial Sequence
  Completely synthetic DNA Sequence
gaatacatgccgatggaaagcgacggggctgaagagatggaggtg

SEQ ID NO: 7 - DNA, 45 bp, Artificial Sequence
  Completely synthetic DNA Sequence
cccctccatctcttcagccccgtcgctttccatcggcatgtattc

SEQ ID NO: 8 - DNA, 36 bp, Artificial Sequence
  Completely synthetic DNA Sequence
gaattcagatctaccatgagcgatgttaccattgtg

SEQ ID NO: 9 - DNA, 30 bp, Artificial Sequence
  Completely synthetic DNA Sequence
tctagatcttattctcgtccacttgcagag

SEQ ID NO: 10 - DNA, 48 bp, Artificial Sequence
  Completely synthetic DNA Sequence
ggtaccatggaatacatgccgatggaaagcgatgttaccattgtgaag

SEQ ID NO: 11 - DNA, 33 bp, Artificial Sequence
  Completely synthetic DNA Sequence
aagcttagatctaccatgaatgaggtgtctgtc

SEQ ID NO: 12 - DNA, 30 bp, Artificial Sequence
  Completely synthetic DNA Sequence
gaattcggatcctcactcgcggatgctggc

SEQ ID NO: 13 - DNA, 49 bp, Artificial Sequence
  Completely synthetic DNA Sequence
ggtaccatggaatacatgccgatggaaaatgaggtgtctgtcatcaaag

SEQ ID NO: 14 - protein, 6 aa, Artificial Sequence
  Completely synthetic Amino Acid Sequence
EYMPME

SEQ ID NO: 15 - protein, 13 aa, Artificial Sequence
  Completely synthetic Amino Acid Sequence
GGRARTSSFAEPG

SEQ ID NO: 16 - protein, 15 aa, Artificial Sequence
  Completely synthetic Amino Acid Sequence
KKGGRARTSSFAEPG